(19) United States
(12) Patent Application Publication
(10) Pub. No.: US 2023/0404926 A1
(43) Pub. Date: Dec. 21, 2023

(54) DELAYED RELEASE SOFTGEL CAPSULES

(71) Applicant: R.P. Scherer Technologies, LLC, Carson City, NV (US)

(72) Inventors: Karunakar Sukuru, St. Petersburg, FL (US); Qi Fang, St. Petersburg, FL (US)

(21) Appl. No.: 18/036,209

(22) PCT Filed: Nov. 10, 2021
(86) PCT No.: PCT/US2021/072326

§ 371 Date: May 10, 2023

Related U.S. Application Data
(60) Provisional application No. 63/112,453, filed on Nov. 11, 2020.

(51) Int. Cl. A61K 9/48; A61K 47/10; A61K 47/36; A61K 47/32; A61K 35/60

(52) U.S. Cl. CPC A61K 9/4816; A61K 9/4833; A61K 35/60; A61K 47/36; A61K 47/32; A61K 47/10

(57) ABSTRACT

Delayed release softgel capsules including a fill material and a pH dependent shell composition. In one embodiments, the pH dependent shell composition includes gelatin, pectin, dextrose, and from about 0.5 wt % to about 10 wt % of a synthetic polymer. In an alternative embodiment, the pH dependent shell composition includes gelatin, pectin, dextrose, and an organic acid. The delayed release nature of the capsules inhibits premature release of the fill material in acidic pHs (such as pH of from any of about 1.2 to about 6).

### CROSS REFERENCE TO RELATED APPLICATION(S)

The present application claims priority to U.S. Provisional Application No. 63/112,453 filed on Nov. 11, 2020, the contents of which are herein incorporated in its entirety.

### FIELD OF THE INVENTION

The present invention relates to delayed release softgel capsules. In certain embodiments, wherein the gelatin-based shell compositions possess delayed release properties with the inclusion of a low amount of synthetic polymer, an organic acid, or a combination thereof.

### BACKGROUND OF THE INVENTION

Soft capsules, in particular, soft gelatin capsules (or softgel capsules), provide a dosage form which is more readily accepted by patients, since the capsules are easy to swallow and need not be flavored in order to mask any unpleasant taste of the active agent. Softgel encapsulation of drugs further provides the potential to improve the bioavailability of the pharmaceutical agents. For example, active ingredients may be rapidly released in liquid form as soon as the gelatin shell ruptures.

Efforts have been made to create delayed release dosage forms. Delayed release dosage forms are designed to protect the contents of the dosage forms from gastric conditions. For example, delayed release dosage forms may be produced by adding a pH dependent coating to the surface of a manufactured dosage form such as a tablet or a capsule. Such coatings may be applied through spraying the dosage form, followed by drying the dosage form, usually at elevated temperatures. This method of coating a capsule with a pH dependent coating may lead to disadvantages in terms of performance and appearance. For example, the capsule may appear rough, the coating may be applied unevenly, and/or the coating can be prone to cracking or flaking off the dosage form. Additionally, the process of applying a pH dependent coating is very inefficient.

Other delayed release dosage forms have been developed in which conventional pH dependent polymers (i.e., acid-insoluble polymers) are added in the capsule shell. However, the addition of conventional pH dependent polymers can lead to capsules that are prone to leaking due to insufficient sealing or that are fragile (i.e., like eggshells) due to the inclusion of a high amount of polymer.

Improving pH dependent shell compositions of softgel capsules is an ongoing endeavor.

### SUMMARY OF THE INVENTION

The present invention is directed to delayed release softgel capsules. The delayed release softgel capsules comprise (a) a fill material and (2) a pH dependent shell composition. The delayed release softgel capsules according to the present invention do not require a pH dependent coating. By eliminating the need to add a pH dependent coating to the softgel capsule, the risk of damaging the capsules during the coating process is also minimized.

In certain embodiments, the pH dependent shell composition comprises: (a) a gelatin, (b) dextrose, (c) a pectin such as a low methoxyl pectin, (d) from about 0.5 wt % to about 10 wt % of a synthetic polymer, based on the total weight of the dried pH dependent shell composition.

In certain embodiments, the pH dependent shell composition comprises: (a) a film former and (b) from about 0.5 wt % to about 10 wt % of a synthetic polymer, based on the total weight of the dried pH dependent shell composition.

In certain embodiments, the pH dependent shell composition comprises: (a) a gelatin, (b) dextrose, (c) a pectin such as a low methoxyl pectin, and (d) an organic acid.

In certain embodiments, the pH dependent shell composition comprises: (a) a film former, and (b) an organic acid.

The present disclosure is also directed to a process of making any of the delayed release softgel capsules described herein.

In certain embodiments, the instant disclosure is also directed to a method of treating a condition by administering to a subject in need thereof any of the delayed release softgel compositions described herein.

The softgel capsules described herein, the pH dependent shell compositions described herein, and their preparation process (e.g., existence or non-existence of a curing step and its conditions, organic acid wash, and so on) may be tuned/adjusted/modified to attain a target pH dissolution/disintegration profile of the shell composition at various pH environments (e.g., rupture/dissolution/disintegration time in acidic medium and in buffer medium).

In certain embodiments, the instant disclosure is directed to methods of inhibiting premature release of a fill material (and correspondingly of an active agent present in the fill material) early in the gastrointestinal tract.

In certain embodiments, the instant disclosure is directed to methods of inhibiting the occurrence of belching due to premature release of a fill material (and correspondingly of an active agent present in the fill material) early in the gastrointestinal tract.

### DETAILED DESCRIPTION OF THE INVENTION

The present invention advances the state of the art by developing delayed release oral dosage forms, in particular, delayed release softgel capsules, that achieve the advantages associated with the conventional delayed release dosage forms without the need to apply a pH dependent coating. The delayed release softgel capsules of the present invention do not dissolve/disintegrate in a gastric environment of the stomach, but rather dissolve at a target pH, e.g., above about 1.2, above about 2, above about 3, above about 3.5, above about 4, above about 5, above about 6, or above about 6.8. The dissolution profile of the delayed release softgel capsules described herein can be tuned by modifying the shell composition of the softgel capsules.

Such mechanism is beneficial for delivery of active ingredients that may cause stomach irritation or are sensitive to the acidic environment of the stomach. Such mechanism is also beneficial for reducing belching after consuming capsules that encapsulate fill materials that tend to contribute to belching. For instance, belching often occurs upon consuming vitamin, minerals, supplements, and/or pharmaceutical products that are formulated in dosage form exhibiting some leaking (even of a very small amount), in the stomach, before reaching the intestines. The leakage can be particularly problematic when the belching is associated with substances that have a noisome perception such as fish oil and garlic that are commonly delivered in softgels. The delayed release softgel capsules described herein may be formulated in a manner that minimizes and/or eliminates premature leakage (and consequently premature release of the capsule's fill) in the gastric environment of the stomach.

### Definitions

As used herein, the term “pH dependent” is used to refer to the dissolution or disintegration resistant property of a substance such that dissolution or disintegration does not occur or does not substantially occur in a gastric environment of the stomach, e.g., for a time period of at least about 15 minutes, at least about 30 minutes, at least about one hour, at least about two hours, at least about three hours, at least about four hours, or at least about five hours. In certain embodiments, the gastric environment of the stomach may be simulated here with 0.1N HCl and optionally with the addition of pepsin adjusted to a pH of 1.2, 2, 3, 4, 5, or 6 with a buffer such as phosphate buffer solution, sodium hydroxide solution, or potassium hydroxide solution. It should be noted that pharmacopeial methods do not include pepsin, however, pepsin was added in certain dissolution/disintegration tests described herein to better simulate/mimic in-vivo conditions. Hence, without being construed as limited, in certain embodiments, the compositions described herein are resistant to dissolution/disintegration for the durations outlined above even at 0.1N HCl environments that include Pepsin (which is presumed to be a more aggressive environment that 0.1N HCl without Pepsin).

For example, the embodiments described herein include a pH dependent shell composition that preferentially dissolves in pH of about 3.5 or higher, 4 or higher, 5 or higher, or 6 or higher (e.g., in biological, artificial or simulated duodenal environment and/or intestinal fluid) as compared to biological, artificial or simulated gastric fluid. In certain embodiments, the intestinal environment may be simulated here with pH 6.8 phosphate buffer with or without Pancreatin. For instance, pH dependent shell composition described herein dissolves in pH of about 3.5 or higher, 4 or higher, 5 or higher, or 6 or higher (e.g., in biological, artificial or simulated duodenal environment and/or intestinal fluid such as pH 6.8 phosphate buffer optionally with Pancreatin) in less than about 60 minutes, less than about 45 minutes, less than about 30 minutes, less than about 20 minutes, less than about 10 minutes, or less than about 5 minutes. It should be noted that pharmacopeial methods do not include pancreatin, however, pancreatin was added in certain dissolution/disintegration tests described herein to better simulate/mimic in-vivo conditions.

Hence, without being construed as limited, in certain embodiments, the compositions described herein exhibit similar dissolution/disintegration profiles at pH of about 3.5 or higher, 4 or higher, 5 or higher, 6 or higher, or of 6.8 buffer environments that include Pancreatin (which is presumed to be a more aggressive environment that pH 6.8 buffer environment without Pancreatin).

As used herein, “pharmaceutically active ingredient,” “active agents” refers to a drug or compound that may be used in the diagnosis, cure, mitigation, treatment, or prevention of a condition. In certain embodiments, suitable “active agents” include nutraceuticals, such as, vitamins, minerals, and supplements (VMS). Exemplary delayed release softgel capsules may include, without limitations, capsules containing lactic acid bacteria, probiotics, fish oil capsules, valproic acid, garlic, peppermint oil, polyethylene glycol, ibuprofen solution or suspension, proton pump inhibitors, aspirin and similar products.

The term “condition” or “conditions” refers to those medical conditions that can be treated or prevented by administration to a subject of an effective amount of an active agent.

As used herein, the term “active ingredient” refers to any material that is intended to produce a therapeutic, prophylactic, or other intended effect, whether or not approved by a government agency for that purpose. This term with respect to a specific agent includes the pharmaceutically active agent, and all pharmaceutically acceptable salts, solvates and crystalline forms thereof, where the salts, solvates and crystalline forms are pharmaceutically active.

Any pharmaceutically active ingredient may be used for purposes of the present invention, including both those that are water-soluble and those that are poorly soluble in water. Suitable pharmaceutically active ingredients include, without limitation, analgesics and anti-inflammatory agents (e.g., ibuprofen, naproxen sodium, aspirin), antacids, anthelmintic, anti-arrhythmic agents, anti-bacterial agents, anti-coagulants, anti-depressants, anti-diabetics, anti-diarrheal, anti-epileptics, anti-fungal agents, anti-gout agents, anti-hypertensive agents, anti-malarial, anti-migraine agents, anti-muscarinic agents, anti-neoplastic agents and immunosuppressants, anti-protozoal agents, anti-rheumatics, anti-thyroid agents, antivirals, anxiolytics, sedatives, hypnotics and neuroleptics, beta-blockers, cardiac inotropic agents, corticosteroids, cough suppressants, cytotoxics, decongestants, diuretics, enzymes, anti-parkinsonian agents, gastro-intestinal agents, histamine receptor antagonists, lipid regulating agents, local anesthetics, neuromuscular agents, nitrates and anti-anginal agents, nutritional agents, opioid analgesics, anticonvulsant agents (e.g., valproic acid), oral vaccines, proteins, peptides and recombinant drugs, sex hormones and contraceptives, spermicides, stimulants, and combinations thereof.

In some embodiments, the active pharmaceutical ingredient may be selected, without limitations, from the group consisting of dabigatran, dronedarone, ticagrelor, iloperidone, ivacaftor, midostaurine, asimadoline, beclomethasone, apremilast, sapacitabine, linsitinib, abiraterone, vitamin D analogs (e.g., calcifediol, calcitriol, paricalcitol, doxercalciferol), COX-2 inhibitors (e.g., celecoxib, valdecoxib, rofecoxib), tacrolimus, testosterone, lubiprostone, pharmaceutically acceptable salts thereof, and combinations thereof.

In some embodiments, the lipids in the dosage form may be selected, without limitations, from the group consisting of almond oil, argan oil, avocado oil, borage seed oil, canola oil, cashew oil, castor oil, hydrogenated castor oil, cocoa butter, coconut oil, colza oil, corn oil, cottonseed oil, grape seed oil, hazelnut oil, hemp oil, hydroxylated lecithin, lecithin, linseed oil, macadamia oil, mango butter, manila oil, mongongo nut oil, olive oil, palm kernel oil, palm oil, peanut oil, pecan oil, perilla oil, pine nut oil, pistachio oil, poppy seed oil, pumpkin seed oil, peppermint oil, rice bran oil, safflower oil, sesame oil, shea butter, soybean oil, sunflower oil, hydrogenated vegetable oil, walnut oil, and watermelon seed oil. Other oil and fats may include, but not be limited to, fish oil (omega-3), krill oil, animal or vegetable fats, e.g., in their hydrogenated form, free fatty acids and mono-, di-, and tri-glycerides with C8-, C10-, C12-, C14-, C16-, C18-, C20- and C22-fatty acids, fatty acid esters like EPA and DHA 3 and combinations thereof.

According to certain embodiments, active agents may include lipid-lowering agents including, but not limited to, statins (e.g., lovastatin, simvastatin, pravastatin, fluvastatin, atorvastatin, rosuvastatin, and pitavastatin), fibrates (e.g, clofibrate, ciprofibrate, bezafibrate, fenofibrate, and gemfibrozil), niacin, bile acid sequestrants, ezetimibe, lomitapide, phytosterols, and the pharmaceutically acceptable salts, hydrates, solvates and prodrugs thereof, mixtures of any of the foregoing, and the like.

Suitable nutraceutical active agents may include, but are not limited to, 5-hydroxytryptophan, acetyl L-carnitine, alpha lipoic acid, alpha-ketoglutarates, bee products, betaine hydrochloride, bovine cartilage, caffeine, cetyl myristoleate, charcoal, chitosan, choline, chondroitin sulfate, coenzyme Q10, collagen, colostrum, creatine, cyanocobalamin (Vitamin 812), dimethylaminoethanol, fumaric acid, germanium sequioxide, glandular products, glucosamine HCl, glucosamine sulfate, hydroxyl methyl butyrate, immunoglobulin, lactic acid, L-Carnitine, liver products, malic acid, maltose-anhydrous, mannose (d-mannose), methyl sulfonyl methane, phytosterols, picolinic acid, pyruvate, red yeast extract, S-adenosylmethionine, selenium yeast, shark cartilage, theobromine, vanadyl sulfate, and yeast.

Suitable nutritional supplement active agents may include vitamins, minerals, fiber, fatty acids, amino acids, herbal supplements or a combination thereof.

Suitable vitamin active agents may include, but are not limited to, the following: ascorbic acid (Vitamin C), B vitamins, biotin, fat soluble vitamins, folic acid, hydroxycitric acid, inositol, mineral ascorbates, mixed tocopherols, niacin (Vitamin B3), orotic acid, para-aminobenzoic acid, panthothenates, panthothenic acid (Vitamin B5), pyridoxine hydrochloride (Vitamin B6), riboflavin (Vitamin B2), synthetic vitamins, thiamine (Vitamin B1), tocotrienols, vitamin A, vitamin D, vitamin E, vitamin F, vitamin K, vitamin oils and oil soluble vitamins.

Suitable herbal supplement active agents may include, but are not limited to, the following: arnica, bilberry, black cohosh, cat's claw, chamomile, echinacea, evening primrose oil, fenugreek, flaxseed, feverfew, garlic oil, ginger root, ginko biloba, ginseng, goldenrod, hawthorn, kava-kava, licorice, milk thistle, psyllium, rauowolfia, senna, soybean, St. John's wort, saw palmetto, turmeric, valerian.

Minerals active agents may include, but are not limited to, the following: boron, calcium, chelated minerals, chloride, chromium, coated minerals, cobalt, copper, dolomite, iodine, iron, magnesium, manganese, mineral premixes, mineral products, molybdenum, phosphorus, potassium, selenium, sodium, vanadium, malic acid, pyruvate, zinc and other minerals.

Examples of other possible active agents include, but are not limited to, antihistamines (e.g., ranitidine, dimenhydrinate, diphenhydramine, chlorpheniramine and dexchlorpheniramine maleate), non-steroidal anti-inflammatory agents (e.g., aspirin, celecoxib, Cox-2 inhibitors, diclofenac, benoxaprofen, flurbiprofen, fenoprofen, flubufen, indoprofen, piroprofen, carprofen, oxaprozin, pramoprofen, muroprofen, trioxaprofen, suprofen, aminoprofen, fluprofen, bucloxic acid, indomethacin, sulindac, zomepirac, tiopinac, zidometacin, acemetacin, fentiazac, clidanac, oxpinac, meclofenamic acid, flufenamic acid, niflumic acid, tolfenamic acid, diflurisal, flufenisal, piroxicam, sudoxicam, isoxicam, aceclofenac, aloxiprin, azapropazone, benorilate, bromfenac, carprofen, choline magnesium salicylate, diflunisal, etodolac, etoricoxib, faislamine, fenbufen, fenoprofen, flurbiprofen, ibuprofen, indometacin, ketoprofen, ketorolac, lomoxicam, loxoprofen, meloxicam, mefenamic acid, metamizole, methyl salicylate, magnesium salicylate, nabumetone, naproxen, nimesulide, oxyphenbutazone, parecoxib, phenylbutazone, salicyl salicylate, sulindac, sulfinpyrazone, tenoxicam, tiaprofenic acid, tolmetin. pharmaceutically acceptable salts thereof and mixtures thereof) and acetaminophen, anti-emetics (e.g., metoclopramide, methylnaltrexone), anti-epileptics (e.g., phenyloin, meprobmate and nitrazepam), vasodilators (e.g., nifedipine, papaverine, diltiazem and nicardipine), anti-tussive agents and expectorants (e.g. codeine phosphate), anti-asthmatics (e.g. theophylline), antacids, anti-spasmodics (e.g. atropine, scopolamine), antidiabetics (e.g., insulin), diuretics (e.g., ethacrynic acid, bendrofluthiazide), anti-hypotensives (e.g., propranolol, clonidine), antihypertensives (e.g., clonidine, methyldopa), bronchodilatiors (e.g., albuterol), steroids (e.g., hydrocortisone, triamcinolone, prednisone), antibiotics (e.g., tetracycline), antihemorrhoidals, hypnotics, psychotropics, antidiarrheals, mucolytics, sedatives, decongestants (e.g. pseudoephedrine), laxatives, vitamins, stimulants (including appetite suppressants such as phenylpropanolamine) and cannabinoids, as well as pharmaceutically acceptable salts, hydrates, solvates, and prodrugs thereof.

The active agent that may also be a benzodiazepine, barbiturate, stimulants, or mixtures thereof. The term “benzodiazepines” refers to a benzodiazepine and drugs that are derivatives of a benzodiazepine that are able to depress the central nervous system.

Benzodiazepines include, but are not limited to, alprazolam, bromazepam, chlordiazepoxide, clorazepate, diazepam, estazolam, flurazepam, halazepam, ketazolam, lorazepam, nitrazepam, oxazepam, prazepam, quazepam, temazepam, triazolam, as well as pharmaceutically acceptable salts, hydrates, solvates, prodrugs and mixtures thereof. Benzodiazepine antagonists that can be used as active agent include, but are not limited to, flumazenil as well as pharmaceutically acceptable salts, hydrates, solvates and mixtures thereof.

The term “barbiturates” refers to sedative-hypnotic drugs derived from barbituric acid (2,4,6,-trioxohexahydropyrimidine). Barbiturates include, but are not limited to, amobarbital, aprobarbotal, butabarbital, butalbital, methohexital, mephobarbital, metharbital, pentobarbital, phenobarbital, secobarbital as well as pharmaceutically acceptable salts, hydrates, solvates, prodrugs, and mixtures thereof. Barbiturate antagonists that can be used as active agent include, but are not limited to, amphetamines as well as pharmaceutically acceptable salts, hydrates, solvates and mixtures thereof.

The term “stimulants” includes, but is not limited to, amphetamines such as dextroamphetamine resin complex, dextroamphetamine, methamphetamine, methylphenidate, as well as pharmaceutically acceptable salts, hydrates, and solvates and mixtures thereof. Stimulant antagonists that can be used as active agent include, but are not limited to, benzodiazepines, as well as pharmaceutically acceptable salts, hydrates, solvates and mixtures thereof.

The dosage forms according to the disclosure include various active agents and their pharmaceutically acceptable salts thereof. Pharmaceutically acceptable salts include, but are not limited to, inorganic acid salts such as hydrochloride, hydrobromide, sulfate, phosphate and the like; organic acid salts such as formate, acetate, trifluoroacetate, maleate, tartrate and the like; sulfonates such as methanesulfonate, benzenesulfonate, p-toluenesulfonate, and the like; amino acid salts such as arginate, asparginate, glutamate and the like, and metal salts such as sodium salt, potassium salt, cesium salt and the like; alkaline earth metals such as calcium salt, magnesium salt and the like; organic amine salts such as triethylamine salt, pyridine salt, picoline salt, ethanolamine salt, triethanolamine salt, dicyclohexylamine salt, N,N′-dibenzylethylenediamine salt and the like.

As used herein, the terms “therapeutically effective” and an “effective amount” refer to the amount of active agent or the rate at which it is administered which is needed to produce a desired therapeutic result.

As used herein, “shell” or “shell composition” refers to the shell of a softgel capsule which encapsulates a fill material.

As used herein, “free or substantially free,” refers to a composition that comprises less than about 1 wt %, less than about 0.5 wt %, less than about 0.25 wt %, less than about 0.1 wt %, less than about 0.05 wt %, less than about 0.01 wt %, or 0 wt % of said component.

All references to wt % throughout the specifications and the claims refer to the weight of the component in reference to the weight of the entire subject composition and may also be designated as w/w.

As used herein, “fill material” or “fill” refers to the composition that is encapsulated by the pH dependent capsule shell and contains at least one pharmaceutically active ingredient.

As used herein, “delayed release capsules” or “delayed release softgel capsules” or “pH dependent capsules” or “pH dependent softgel capsules” refer to capsules which have delayed or pH dependent properties once the fill material is encapsulated in the shell, and the capsules are dried. In certain embodiments, these terms may refer to capsules that have also been cured after drying. In certain embodiments, no further processing steps past drying are required. In certain embodiments, no further processing steps past curing are required.

As used herein, “about” refers to any values that are within a variation of ±10%, such that “about 10” would include from 9 to 11. As used herein, “a,” “an,” or “the” refers to one or more, unless otherwise specified. Thus, for example, reference to “an excipient” includes a single excipient as well as a mixture of two or more different excipients, and the like.

Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context.

The use of any and all examples, or exemplary language (e.g., “such as”) provided herein, is intended merely to illuminate certain materials and methods and does not pose a limitation on scope. No language in the specification should be construed as indicating any non-claimed element as essential to the practice of the disclosed materials and methods.

### Softgel Capsule Dosage Form

According to a first embodiment, a pH dependent softgel capsule comprises (a) a fill material and (b) a pH dependent shell composition, wherein the fill material comprises at least one active agent, wherein the pH dependent shell composition comprises a gelatin, dextrose, a pH dependent material (e.g., a low methoxyl pectin), a synthetic polymer, and optionally a plasticizer. Preferably, the synthetic polymer is present in the pH dependent shell composition in an amount of about 0.5 wt % to about 10 wt %, based on the total weight of the dried pH dependent shell composition.

According to certain embodiments, a pH dependent softgel capsule comprises (a) a fill material and (b) a pH dependent shell composition, wherein the fill material comprises at least one active agent, wherein the pH dependent shell composition comprises a film former and a synthetic polymer. Preferably, the synthetic polymer is present in the pH dependent shell composition in an amount of about 0.5 wt % to about 10 wt %, based on the total weight of the dried pH dependent shell composition. The pH dependent shell composition may further include at least one of pectin, dextrose, or gelatin.

According to alternative embodiments, a pH dependent softgel capsule comprises (a) a fill material and (b) a pH dependent shell composition, wherein the fill material comprises at least one active agent, wherein the pH dependent shell composition comprises a gelatin, dextrose, a pH dependent material (e.g., a low methoxyl pectin), an organic acid, and optionally a plasticizer.

According to alternative embodiments, a pH dependent softgel capsule comprises (a) a fill material and (b) a pH dependent shell composition, wherein the fill material comprises at least one active agent, wherein the pH dependent shell composition comprises a film former and an organic acid. The pH dependent shell composition may further include at least one of pectin, dextrose, or gelatin.

According to certain embodiments, a pH dependent softgel capsule comprises (a) a fill material and (b) a pH dependent shell composition, wherein the fill material comprises at least one pharmaceutically active ingredient, wherein the pH dependent shell composition comprises a gelatin, dextrose, a pH dependent material (e.g., a low methoxyl pectin), an organic acid, a synthetic polymer, and optionally a plasticizer. Preferably, the synthetic polymer is present in the pH dependent shell composition in an amount of about 0.5 wt % to about 10 wt %, based on the total weight of the dried pH dependent shell composition.

According to certain embodiments, a pH dependent softgel capsule comprises (a) a fill material and (b) a pH dependent shell composition, wherein the fill material comprises at least one pharmaceutically active ingredient, wherein the pH dependent shell composition comprises a film former, an organic acid, and a synthetic polymer. Preferably, the synthetic polymer is present in the pH dependent shell composition in an amount of about 0.5 wt % to about 10 wt %, based on the total weight of the dried pH dependent shell composition. The pH dependent shell composition may further include at least one of pectin, dextrose, or gelatin.

Suitable fill materials comprise at least one pharmaceutically active ingredient and can be made according to known methods. In addition to the at least one pharmaceutically active ingredient, suitable fill materials may comprise additional fill components such as flavoring agents, sweetening agents, coloring agents and fillers or other pharmaceutically acceptable excipients or additives such as synthetic dyes and mineral oxides. Suitable amounts of pharmaceutically active ingredient and pharmaceutically acceptable excipients can be readily determined by one of ordinary skill in the art.

In an embodiment, the gelatin in the pH dependent shell composition may include Type A gelatin, Type B gelatin, a hide or skin gelatin (e.g., calf skin, pig skin) and/or a bone gelatin (e.g., bovine bone, pig bone) used alone or in combination. In one embodiment, the gelatin is a 250 Bloom gelatin. In another embodiment, there is only one type of gelatin. In yet another embodiment, the gelatin is a combination of at least two types of gelatins. In an embodiment, the amount of gelatin in the pH dependent shell composition is from about 30 wt % to about 85 wt %, from about 30 wt % to about 75 wt %, from about 30 wt % to about 65 wt %, from about 30 wt % to about 55 wt %, from about 30 wt % to about 40 wt %, about 40 wt % to about 80 wt %, from about 45 wt % to about 65 wt %, from about 45 wt % to about 75 wt %, or from about 50 wt % to about 70 wt %, or any single value or sub-range therein, based on total weight of the dry capsule shell composition.

In certain embodiments, the pH dependent shell composition may include instead or in addition to at least one of: gelatin, pectin, or dextrose, a film former that is a non-animal derived gelling agent. Suitable non-animal derived gelling agents include, without limitations, carrageenan, starch, pregelatinized starch, xanthan gum, agar, pectin, alginate, sugar, high molecular weight polyethylene glycol, sugar derived alcohol, a cellulose derivative, a cellulosic polymer, hydroxyethylcellulose, hydroxypropylcellulose, hydroxypropylmethylcellulose, carboxymethylcellulose, microcrystalline cellulose, attapulgite, bentonite, dextrin, alginate, kaolin, lecithin, magnesium aluminum silicate, carbomer, carbopol, silicon dioxide, curdlan, furcelleran, albumin, soy protein, chitosan, or a combination thereof.

The carrageenan can be at least one of iota carrageenan, kappa carrageenan and lambda carrageenan.

The starch can be modified starch or native starch, sweet potato starch, potato starch, corn starch, tapioca starch, pea starch, hydroxy propylated starch, hydroxyalkylated starch, acid-treated starch, dextrin, high amylose non-modified corn starch, modified waxy maize starch, non-granular starch, modified high amylose corn starch, pregelatinized rice flour and a combination thereof. As used herein and in the claims, the term “modified starch” includes such starches as hydroxypropylated starches, acid thinned starches and the like. In general, modified starches are products prepared by chemical treatment of starches, for example, acid treatment starches, enzyme treatment starches, oxidized starches, cross-bonding starches, and other starch derivatives. It is preferred that the modified starches be derivatized wherein side chains are modified with hydrophilic or hydrophobic groups to thereby form a more complicated structure with a strong interaction between side chains.

In certain embodiments, the non-animal gelling agent is in the shell composition in an amount, e.g., of about 2 wt. % to about 20 wt. %, about 2 wt. % to about 15 wt. %, about 2 wt. % to about 40 wt. %, about 10 wt. % to about 80 wt. %, or about 15 wt. % to about 75 wt. %, or about 20 wt. % to about 70 wt. %, or about 25 wt. % to about 60 wt. %, or about 25 wt. % to about 45 wt. %, or about 20 wt. % to about 35 wt. %, or about 30 wt. % to about 40 wt. %, or about 32 wt. %, or about 35 wt. %, or about 38 wt. %, or any sub-range or single concentration value therein, with all wt. % being based on the total weight of the shell composition. In one embodiment, the non-animal gelling agent includes carrageenan and does not include starch (or modified starch). In one embodiment, the softgel shell composition is substantially free or free of starch (or modified starch).

In one embodiment, the pH dependent capsule shell composition comprises dextrose. In an embodiment, the amount of dextrose in the pH dependent capsule shell composition is from about 0.001 wt % to about 1.0 wt %, from about 0.002 wt % to about 0.008 wt %, from about 0.005 wt % or about 0.01 wt % to about 4 wt %, from about 0.1 wt % or about 0.15 wt % to about 3 wt %, from about 0.1 wt % to about 1 wt %, from about 0.1 or about 0.15 wt % or about 0.2 wt % or about 0.25 wt % to about 2 wt %, from about 0.1 wt % to about 0.2 wt %, from about 0.1 wt % to about 0.4 wt %, or any single value or sub-range therein, based on total weight of the dry capsule shell composition. The dextrose may be added to the delayed release capsule shell to mitigate potential reduction in gel strength. Without being construed as limiting, it is believed that the dextrose interacts with the gelatin in the shell composition and cause the gelatin to cross-link. The concentration of dextrose in the pH dependent shell composition may be in an effective amount to improve the gel strength but not so high that it would interfere with the seal of the capsule or manufacturability or the product performance.

In some embodiments, the pH dependent shell composition may comprise pectin, e.g., a low methoxyl pectin. In an embodiment, the pectin is low methylester (LM) pectin with Degree of Esterification lower than 50. In some embodiments, the pectin is amidated pectin. In certain embodiments, the amidated pectin may have a Degree of Amidation of lower than 25, from 5 to 25, from 10 to 20, or from 15 to 25. In other embodiments, the low methoxyl (LM) pectin is non-amidated pectin. In certain embodiments, the pectin is a combination of amidated pectin and non-amidated pectin. The addition of pectin contributes to the pH dependent nature of the dosage form.

Too much pectin in the dosage form may reduce the gel strength of the softgel capsule which may in turn adversely affect the sealability of the softgel capsule. Too much pectin in the pH dependent shell composition may also increase the viscosity of the shell composition, making it challenging or impossible to process from a manufacturing standpoint. Therefore, pectin may be added to the dosage form at a concentration that is sufficiently high to form a delayed release dosage form and at the same time is sufficiently low to mitigate the reduction in gel strength and to mitigate viscosity increase.

In an embodiment, an amount of pectin in the pH dependent shell composition is about 2 wt % to about 20 wt %, from about 3 wt % to about 15 wt %, from about 3 wt % to about 5.5 wt %, from about 4 wt % to about 11 wt %, from about 7 wt % to about 12 wt %, from about 8 wt % to about 13 wt %, or from about 5 wt % to about 10 wt %, or any single value or sub-range therein, based on total weight of the dry capsule shell composition.

The degree of esterification of the pectin incorporated in the pH dependent shell composition may be lower than about 50%, or may range from about 10% to about 50%, from about 20% to about 40%, or from about 25% to about 35%. Also, the pectin may be amidated or non-amidated.

In certain embodiments, the pH dependent shell composition comprises a stabilizer and/or a binder comprising gellan gum. In certain embodiments, the amount of stabilizer and/or binder (e.g., gellan gum) in the pH dependent shell composition is about 0.05 wt % to about 5 wt %, about 0.1 wt % to about 3 wt %, or about 0.2 wt % to about 2 wt % of stabilizer and/or binder (e.g., gellan gum), or any single value or sub-range therein, based on total weight of the dry capsule shell composition.

In certain embodiments, the pH dependent shell composition may have a viscosity ranging from any of about 20,000 cPs, about 30,000 cPs, about 40,000 cPs, about 50,000 cPs, about 60,000 cPs, or about 70,000 cPs to any of about 80,000 cPs, about 90,000 cPs, about 100,000 cPs, about 110,000 cPs, about 120,000 cPs, about 130,000 cPs, about 140,000 cPs, or about 150,000 cPs, or any sub-range or single value therein. In one embodiment, the pH dependent shell composition has a viscosity ranging from about 100,000 cPs to about 130,000 cPs, or from about 110,000 cPs to about 125,000 cPs, or about 115,000 cPs, or about 120,000 cPs. The viscosity is measured using a rheometer at 60° C. A gel mass sample (e.g., of any of the pH dependent shell compositions described herein) is loaded onto the platform of the rheometer, maintained at 60° C. A disc rotates at a certain speed to provide a fixed shear rate. The viscosity is obtained by measuring the shear stress and shear rate.

In certain embodiments, the pH dependent shell composition may maintain a viscosity that is suitable for manufacturability even after being aged in heat for up to about 24 hours, up to about 48 hours, up to about 72 hours, up to about 96 hours, or up to about 1 week. In certain embodiments, the viscosity of the pH dependent shell composition, after aging in heat (for up to about 24 hours, up to about 48 hours, up to about 72 hours, up to about 96 hours, or up to about 1 week) may reduce (from the viscosity value of the composition prior to aging) by up to about 80%, up to about 70%, up to about 60%, up to about 50%, up to about 40%, up to about 35%, or up to about 30%.

In an embodiment, the plasticizer in the pH dependent shell composition may include glycerin, sorbitol or sorbitol sorbitan solution and combinations thereof. Other suitable plasticizers may include, but not be limited to, sugar alcohol plasticizer such as isomalt, maltitol, xylitol, erythritol, adonitol, dulcitol, pentaerythritol, or mannitol; or polyol plasticizer such as diglycerin, dipropylene glycol, a polyethylene glycol up to 10,000 MW, neopentyl glycol, propylene glycol, 1,3-propanediol, 2-methyl-1,3-propanediol, trimethylolpropane, a polyether polyol, ethanol amines; and mixtures thereof. Other exemplary plasticizers may also include, without limitations, low molecular weight polymers, oligomers, copolymers, oils, small organic molecules, low molecular weight polyols having aliphatic hydroxyls, ester-type plasticizers, glycol ethers, poly(propylene glycol), multi-block polymers, single block polymers, citrate ester-type plasticizers, and triacetin. Such plasticizers may include 1,2-butylene glycol, 2,3-butylene glycol, styrene glycol, monopropylene glycol monoisopropyl ether, propylene glycol monoethyl ether, ethylene glycol monoethyl ether, diethylene glycol monoethyl ether, sorbitol or sorbitol sorbitan solution lactate, ethyl lactate, butyl lactate, ethyl glycolate, dibutyl sebacate, acetyltributylcitrate, triethyl citrate, glyceryl monostearate, polysorbate 80, acetyl triethyl citrate, tributyl citrate and allyl glycolate, and mixtures thereof.

In an embodiment, the amount of plasticizer in the pH dependent shell composition is from about 15 wt % to about 45 wt %, from about 15 wt % to about 40 wt %, from about 18 wt % to about 45 wt %, from about 18 wt % to about 42 wt %, from about 20 wt % to about 35 wt %, from about 25 wt % to about 30 wt %, or any single value, or sub-range therein, based on total weight of the dry capsule shell composition.

In certain embodiments, any of the pH dependent shell compositions described herein may further include a synthetic polymer. Suitable synthetic polymers include, without limitations, acrylic and methacrylic acid polymers, which may be available under the tradename EUDRAGIT®, methacrylic acid-ethyl acrylate copolymer, which may be available under the tradename Kollicoat® and other conventional acid insoluble polymers, e.g., methyl acrylate-methacrylic acid copolymers. Other suitable acid insoluble polymers include, without limitation, cellulose acetate succinate, cellulose acetate phthalate, cellulose acetate butyrate, hydroxypropyl methyl cellulose phthalate, hydroxy propyl methyl cellulose acetate succinate (hypermellose acetate succinate), polyvinyl acetate phthalate (PVAP), alginic acid salts such as sodium alginate and potassium alginate, stearic acid, and shellac.

In certain embodiments, suitable synthetic polymers are water insoluble, such as methacrylic acid-ethyl acrylate copolymer. Adding a water insoluble polymer to the pH dependent shell composition is believed to make the pH dependent shell composition more hydrophobic. When the pH dependent shell composition becomes more hydrophobic (as compared to if the pH dependent shell composition does not include the synthetic polymer), it is believed to reduce the amount of water that migrates from the fill material into the shell composition. This in turn enhances the robustness of the shell composition and allows the shell composition to retain its mechanical strength. This is also believed to enable inhibition of premature release from softgel capsules (that includes said pH dependent shell composition) without having to subject the softgel capsule to an extended curing (e.g., at about 40° C. for 4-5 days). This benefit may be observed even in softgel capsules in which the pH dependent shell composition includes non-amidated pectin. This benefit may also be observed in softgel capsules in which the pH dependent shell composition does not include a stabilizer/binder such as gellan gum. It is also believed that methacrylic acid-ethyl acrylate copolymers (and other suitable acrylate polymers as appreciated by those skilled in the art) in combination with pectin extend the pH performance of the pH dependent shell composition and correspondingly of the softgel capsule (e.g., by extending the durability of the softgel capsules at higher pH values and enabling targeted release of the fill material into a target location within the gastrointestinal tract).

In one embodiment, the synthetic polymer is Kollicoat MAE-100P, which is a methacrylic acid-ethyl acrylate copolymer (1:1). This synthetic polymer may be chosen, in certain embodiments, since it is already pre-neutralized and does not require the addition of a base (such as ammonia) to neutralize or solubilize the polymer during processing.

In certain embodiments, the amount of synthetic polymer in the pH dependent shell compositions described herein is from about 0.5 wt % to about 10 wt %, from about 1 wt. % to about 5 wt. %, from about 1.5 wt. % to about 4 wt. %, or from about 2 wt. % to about 3 wt. %, or any single value, or sub-range therein, based on total weight of the dry capsule shell composition.

The synthetic polymer is believed, without being construed as limiting, to function as a sealant to stop/inhibit the seepage of a fill material from a capsule seal.

In certain embodiments, any of the pH dependent shell compositions described herein may further include an organic acid. Suitable organic acids include lactic acid, tannic acid, citric acid, acetic acid, or a combination thereof. In one embodiment, the organic acid in the pH dependent shell composition comprises lactic acid. In one embodiment, the organic acid in the pH dependent shell composition comprises tannic acid. In one embodiment, the organic acid in the pH dependent shell composition comprises lactic acid and tannic acid.

In certain embodiments, the amount of organic acid in the pH dependent shell compositions described herein is from about 0.1 wt % to about 8 wt %, from about 0.2 wt. % to about 5 wt. %, or from about 0.2 wt. % to about 2 wt. % or any single value, or sub-range therein, based on total weight of the dry capsule shell composition.

The organic acid(s) are believed, without being construed as limiting, to facilitate the interaction between gelatin and pectin to form a more robust softgel capsule.

In certain embodiments, the amount of the various components (e.g., pectin, dextrose, gelatin, synthetic polymer, plasticizer, stabilizer/binder) and the ratio of the various components are tuned to control the dissolution and/or disintegration properties of the softgel capsule across various pH ranges.

For instance, the gelatin to pectin w:w ratio in the pH dependent shell composition may range from any of about 2:1, about 3:1, about 4:1, about 5:1, about 6:1, about 7:1, about 8:1, or about 9:1 to any of about 10:1, about 11:1, about 12:1, about 13:1, about 14:1, about 15:1, about 16:1, about 17:1, about 18:1, about 19:1, or about 20:1, or any sub-range or single value therein. In certain embodiments, lower gelatin to pectin w:w ratios provide for a pH dependent shell composition that is more stable (dissolves/disintegrates slower if at all) in acidic medium (e.g., 0.1N HCl optionally with Pepsin, adjusted to pH with phosphate buffer, sodium hydroxide, or potassium hydroxide), while higher gelatin to pectin w:w ratios provide for a pH dependent shell composition that is less stable (dissolves/disintegrates faster) in acidic medium (e.g., 0.1N HCl optionally with Pepsin, adjusted to pH with phosphate buffer, sodium hydroxide, or potassium hydroxide). The gelatin to pectin w:w ratio may be tuned to attain a particular dissolution/disintegration time for softgel capsule in an acidic medium with a certain pH (e.g., at least about 15 minutes, at least about 30 minutes, at least about 45 minutes, at least about 60 minutes, at least about 90 minutes, or at least about 120 minutes at a pH of 1.2, 2, 3, 4, 5, 6, or a sub-range therein, and so on) and/or a particular dissolution/disintegration time for the softgel capsule in buffer medium with a certain pH (e.g., up to about 5 minutes, up to about 10 minutes, up to about 20 minutes, up to about 30 minutes, up to about 45 minutes, or up to about 60 minutes in biological, artificial or simulated duodenal environment and/or intestinal fluid such as pH 6.8 phosphate buffer, sodium hydroxide buffer, or potassium hydroxide buffer, optionally with Pancreatin.

The gelatin to plasticizer w:w ratio in the pH dependent shell composition may also be tuned to attain a particular capsule hardness level and may range from about 5:1 to about 1:5, from about 4:1 to about 1:4, from about 3:1 to about 1:3, from about 2:1 to about 1:2, about 1:1, or any single ratio value or sub-range therein.

In certain embodiments, the w:w ratio of pectin to stabilizer and/or binder (e.g., gellan gum) is about 1:10 to about 50:1; about 1:5 to about 40:1; about 1:1 to about 25:1 or about 10:1 to about 24:1, or any single ratio value or sub-range therein.

In certain embodiments, the w:w ratio of synthetic polymer to pectin in the pH dependent shell composition is about 3:1 to about 1:20, about 3:1 to about 1:15, from about 3:1 to about 1:10, from about 2:1 to about 1:5, from about 2:1 to about 1:3, about 1:1, or any single ratio value or sub-range therein.

In certain embodiments, the w:w ratio of synthetic polymer to gelatin in the pH dependent shell composition is about 1:3 to about 1:100, about 1:3 to about 1:50, about 1:3 to about 1:25, about 1:3 to about 1:20, about 1:3 to about 1:15, about 1:3 to about 1:10, or about 1:3 to about 1:5, or any single ratio value or sub-range therein.

In certain embodiments, the w:w ratio of organic acid to pectin in the pH dependent shell composition is about 2:1 to about 1:60, about 2:1 to about 1:40, about 2:1 to about 1:20, about 2:1 to about 1:15, about 2:1 to about 1:10, about 1:1 to about 1:5, or any single ratio value or sub-range therein.

In certain embodiments, the w:w ratio of organic acid to gelatin in the pH dependent shell composition is about 1:15 to about 1:250, about 1:15 to about 1:200, about 1:15 to about 1:150, about 1:15 to about 1:100, about 1:20 to about 1:75, about 1:20 to about 1:50, or about 1:30 to about 1:50, or any single ratio value or sub-range therein.

In certain embodiments, the softgel capsules made using the pH dependent shell compositions described herein may have a hardness ranging from any of about 5 N, about 6 N, about 7 N, about 8 N, about 9 N, or about 10 N to any of about 11 N, about 12 N, about 13 N, about 14 N, or about 15 N. The capsule hardness is determined using a hardness tester. The force required to cause a 2.0 mm deformation of the capsule in Newton is defined as the capsule hardness.

In certain embodiments, the softgel capsules made using the pH dependent shell compositions described herein may have a shell moisture ranging from any of about 5%, about 6%, about 7%, about 8%, about 9%, or about 10% to any of about 11%, about 12%, about 13%, about 14%, or about 15%. The shell moisture is determined by loss on drying method. A pH dependent capsule shell composition sample of 1 to 2 grams is placed into a 105° C. oven for 17 hours. The initial weight of the sample is recorded. After drying the sample in the oven at 105° C. for 17 hours, the final weight of the sample is recorded. The percentage of weight loss, calculated in accordance with the below equation, is defined as the shell moisture:

$$\% weight lost = \frac{( initial weight ) - ( final weight )}{( initial weight )} \cdot 100 \%$$

In certain embodiments, the pH dependent shell compositions described herein may have an equilibrium relative humidity ranging from any of about 25%, about 28%, about 30%, about 32%, about 34%, or about 35% to any of about 38%, about 40%, about 42%, about 45%, or about 50%. Equilibrium Relative Humidity (%) is defined as the humidity condition at which the capsule maintained a constant total weight. It is determined using environmental chambers maintained at constant humidity using saturated salt solutions.

In certain embodiments, the softgel capsules made using the pH dependent shell compositions described herein may have a burst strength ranging from any of about 50 kg, about 60 kg, about 70 kg, about 80 kg, or about 90 kg to any of about 100 kg, about 110 kg, about 120 kg, about 130 kg, about 140 kg, or about 150 kg. Burst strength is determined using a texture analyzer. The texture analyzer compressed the capsule until the capsule burst. The force, in kilograms, required to make the capsule burst is defined as burst strength.

In an embodiment, the pH dependent shell composition and the pH dependent softgel capsule may be free or substantially free of a pH dependent overcoat over the softgel shell.

In an embodiment, the pH dependent shell composition and the pH dependent softgel capsule may include divalent cation salts, such as Ca⁺⁺ (e.g., CaCl₂)) or Mg⁺⁺ (e.g., MgCl₂). In another embodiment, the pH dependent shell composition and the pH dependent softgel capsule may be free or substantially free of divalent cation salts, such as Ca⁺⁺ (e.g., CaCl₂)) or Mg⁺⁺ (e.g., MgCl₂). In a further embodiment, the pH dependent shell composition may not include the step of the addition of divalent cation salts, such as Ca⁺⁺ (e.g., CaCl₂)) or Mg⁺⁺ (e.g., MgCl₂) other than an amount of divalent cation salts that me be present in other components.

In an embodiment, the pH dependent shell composition may optionally comprise additional agents such as stabilizers or binders (e.g., gellan gum), coloring agents, flavorings agents, sweetening agents, fillers, antioxidants, diluents, pH modifiers or other pharmaceutically acceptable excipients or additives such as synthetic dyes and mineral oxides.

Exemplary suitable coloring agents may include, but not be limited to, colors such as e.g., white, black, yellow, blue, green, pink, red, orange, violet, indigo, and brown. In specific embodiments, the color of the dosage form can indicate the contents (e.g., one or more active ingredients) contained therein.

Exemplary suitable flavoring agents may include, but not be limited to, “flavor extract” obtained by extracting a part of a raw material, e.g., animal or plant material, often by using a solvent such as ethanol or water; natural essences obtained by extracting essential oils from the blossoms, fruit, roots, etc., or from the whole plants.

Additional exemplary flavoring agents that may be in the dosage form may include, but not be limited to, breath freshening compounds like menthol, spearmint, and cinnamon, coffee beans, other flavors or fragrances such as fruit flavors (e.g., cherry, orange, grape, etc.), especially those used for oral hygiene, as well as actives used in dental and oral cleansing such as quaternary ammonium bases. The effect of flavors may be enhanced using flavor enhancers like tartaric acid, citric acid, vanillin, or the like.

Exemplary sweetening agents may include, but not be limited to, one or more artificial sweeteners, one or more natural sweeteners, or a combination thereof. Artificial sweeteners include, e.g., acesulfame and its various salts such as the potassium salt (available as Sunett®), alitame, aspartame (available as NutraSweet@ and Equal®), salt of aspartame-acesulfame (available as Twinsweet®), neohesperidin dihydrochalcone, naringin dihydrochalcone, dihydrochalcone compounds, neotame, sodium cyclamate, saccharin and its various salts such as the sodium salt (available as Sweet'N Low®), Stevia, chloro derivatives of sucrose such as sucralose (available as Kaltame® and Splenda®), and mogrosides. Natural sweeteners include, e.g., glucose, dextrose, invert sugar, fructose, sucrose, glycyrrhizin; monoammonium glycyrrhizinate (sold under the trade name MagnaSweet®); Stevia rebaudiana (Stevioside), natural intensive sweeteners, such as Lo Han Kuo, polyols such as sorbitol or sorbitol sorbitan solution, mannitol, xylitol, erythritol, and the like.

In an embodiment, the pH dependent shell composition comprises: (a) gelatin, (b) dextrose, (c) a pH dependent polymer (e.g., pectin such as a low methoxyl pectin), (d) from about 0.5 wt % to about 10 wt % of a synthetic polymer, based on total weight of the dried pH dependent shell composition, (e) optionally an organic acid, (f) optionally a plasticizer (e.g., glycerin, sorbitol or sorbitol sorbitan solution, and combinations thereof), and optionally (g) a stabilizer and/or binder (e.g., gellan gum). The amounts and wt:wt ratios of these components may be in accordance with any of the values or ranges described hereinabove.

In an embodiment, the pH dependent shell composition consists essentially of: (a) gelatin, (b) dextrose, (c) a pH dependent polymer (e.g., pectin such as a low methoxyl pectin), (d) from about 0.5 wt % to about 10 wt % of a synthetic polymer, based on total weight of the dried pH dependent shell composition, (e) optionally an organic acid, (f) optionally a plasticizer (e.g., glycerin, sorbitol or sorbitol sorbitan solution, and combinations thereof), and optionally (g) a stabilizer and/or binder (e.g., gellan gum). The amounts and wt:wt ratios of these components may be in accordance with any of the values or ranges described hereinabove.

In an embodiment, the pH dependent shell composition consists of (a) gelatin, (b) dextrose, (c) a pH dependent polymer (e.g., pectin such as a low methoxyl pectin), (d) from about 0.5 wt % to about 10 wt % of a synthetic polymer, based on total weight of the dried pH dependent shell composition, (e) optionally an organic acid, (f) optionally a plasticizer (e.g., glycerin, sorbitol or sorbitol sorbitan solution, and combinations thereof), and optionally (g) a stabilizer and/or binder (e.g., gellan gum). The amounts and wt:wt ratios of these components may be in accordance with any of the values or ranges described hereinabove.

### Dissolution and Disintegration

Reference to a “dissolution” or a “dissolution test” throughout this disclosure refers results from tests performed with a USP Apparatus II with paddles at from any of about 50 RPM to any of about 250 RPM, from any of about 500 ml to any of about 900 ml 0.1N HCL acidic media adjusted to pH 1.2, 2.0, 3.0, 4.0, 5.0, and 6.0 with phosphate buffer solution, sodium hydroxide solution, or potassium hydroxide solution (also referred to as “Acid Stage”). After two hours, phosphate buffer solution, sodium hydroxide solution, or potassium hydroxide solution is added to adjust the pH to 6.8 (also referred to as “pH 6.8 Buffer”). The term “dissolve” with respect to the performance of the softgel capsule and/or shell composition in a two stage dissolution test may be used interchangeably with the term “rupture.” The “two stage dissolution test” may also be referred to herein as a “two stage enteric dissolution test” or as an “enteric dissolution test.”

Reference to a “disintegration” or a “disintegration test” throughout this disclosure refers to results from tests performed with a USP disintegration apparatus in from any of about 500 ml to any of about 900 ml 0.1N HCL acidic media adjusted to pH 1.2, 2.0, 3.0, 4.0 5.0, and 6.0 phosphate buffer solution, sodium hydroxide solution, or potassium hydroxide solution (also referred to as “Acid Stage”). After two hours, phosphate buffer solution, sodium hydroxide solution, or potassium hydroxide solution is added to adjust the pH to 6.8 (also referred to as “pH 6.8 Buffer”). The term “disintegrate” with respect to the performance of the softgel capsule and/or shell composition in a two stage disintegration test may be used interchangeably with the term “rupture.” The “two stage disintegration test” may also be referred to herein as a “two stage enteric disintegration test” or as an “enteric disintegration test.”

In certain embodiments, the shell composition does not dissolve at a pH of 1.2 at 15 minutes, 30 minutes, 45 minutes, 60 minutes, 90 minutes or 120 minutes (e.g., when measured with a USP Apparatus II with paddles at from any of about 50 RPM to any of about 250 RPM in from any of about 500 ml to any of about 900 ml 0.1N HCL acidic media adjusted to pH with phosphate buffer solution, sodium hydroxide solution, or potassium hydroxide solution).

In certain embodiments, the shell composition does not dissolve at a pH of 1.2 for a time period of at least about 15 minutes, at least about 30 minutes, at least about 45 minutes, at least about 60 minutes, at least about 90 minutes, or at least about 120 minutes (e.g., when measured with a USP Apparatus II with paddles at from any of about 50 RPM to any of about 250 RPM in from any of about 500 ml to any of about 900 ml 0.1N HCL acidic media adjusted to pH with phosphate buffer solution, sodium hydroxide solution, or potassium hydroxide solution).

In certain embodiments, the shell composition does not dissolve at a pH of 1.2 for a time period of about 15 minutes to about 360 minutes, about 30 minutes to about 240 minutes, or about 45 minutes to about 180 minutes (e.g., when measured with a USP Apparatus II with paddles at from any of about 50 RPM to any of about 250 RPM in from any of about 500 ml to any of about 900 ml 0.1N HCL acidic media adjusted to pH with phosphate buffer solution, sodium hydroxide solution, or potassium hydroxide solution).

In certain embodiments, the shell composition does not disintegrate at a pH of 1.2 at 15 minutes, 30 minutes, 45 minutes, 60 minutes, 90 minutes or 120 minutes (e.g., when measured with a USP disintegration apparatus in from any of about 500 ml to any of about 900 ml 0.1N HCL acidic media adjusted to pH with phosphate buffer solution, sodium hydroxide solution, or potassium hydroxide solution).

In certain embodiments, the shell composition does not disintegrate at a pH of 1.2 for a time period of at least about 15 minutes, at least about 30 minutes, at least about 45 minutes, at least about 60 minutes, at least about 90 minutes, or at least about 120 minutes (e.g., when measured with a USP disintegration apparatus in from any of about 500 ml to any of about 900 ml 0.1N HCL acidic media adjusted to pH with phosphate buffer solution, sodium hydroxide solution, or potassium hydroxide solution).

In certain embodiments, the shell composition does not disintegrate at a pH of 1.2 for a time period of about 15 minutes to about 360 minutes, about 30 minutes to about 240 minutes, or about 45 minutes to about 180 minutes (e.g., when measured with a USP disintegration apparatus in from any of about 500 ml to any of about 900 ml 0.1N HCL acidic media adjusted to pH with phosphate buffer solution, sodium hydroxide solution, or potassium hydroxide solution).

In certain embodiments, the shell composition does not dissolve at a pH of between 1.2 and 2 at 15 minutes, 30 minutes, 45 minutes, 60 minutes, 90 minutes or 120 minutes (e.g., when measured with a USP Apparatus II with paddles at from any of about 50 RPM to any of about 250 RPM in from any of about 500 ml to any of about 900 ml 0.1N HCL acidic media adjusted to pH with phosphate buffer solution, sodium hydroxide solution, or potassium hydroxide solution).

In certain embodiments, the shell composition does not dissolve at a pH of between 1.2 and 2 for a time period of at least about 15 minutes, at least about 30 minutes, at least about 45 minutes, at least about 60 minutes, at least about 90 minutes, or at least about 120 minutes (e.g., when measured with a USP Apparatus II with paddles at from any of about 50 RPM to any of about 250 RPM in from any of about 500 ml to any of about 900 ml 0.1N HCL acidic media adjusted to pH with phosphate buffer solution, sodium hydroxide solution, or potassium hydroxide solution).

In certain embodiments, the shell composition does not dissolve at a pH of between 1.2 and 2 for a time period of about 15 minutes to about 360 minutes, about 30 minutes to about 240 minutes, or about 45 minutes to about 180 minutes (e.g., when measured with a USP Apparatus II with paddles at from any of about 50 RPM to any of about 250 RPM in from any of about 500 ml to any of about 900 ml 0.1N HCL acidic media adjusted to pH with phosphate buffer solution, sodium hydroxide solution, or potassium hydroxide solution).

In certain embodiments, the shell composition does not disintegrate at a pH of between 1.2 and 2 at 15 minutes, 30 minutes, 45 minutes, 60 minutes, 90 minutes or 120 minutes (e.g., when measured with a USP disintegration apparatus in from any of about 500 ml to any of about 900 ml 0.1N HCL acidic media adjusted to pH with phosphate buffer solution, sodium hydroxide solution, or potassium hydroxide solution).

In certain embodiments, the shell composition does not disintegrate at a pH of between 1.2 and 2 for a time period of at least about 15 minutes, at least about 30 minutes, at least about 45 minutes, at least about 60 minutes, at least about 90 minutes, or at least about 120 minutes (e.g., when measured with a USP disintegration apparatus in from any of about 500 ml to any of about 900 ml 0.1N HCL acidic media adjusted to pH with phosphate buffer solution, sodium hydroxide solution, or potassium hydroxide solution).

In certain embodiments, the shell composition does not disintegrate at a pH of between 1.2 and 2 for a time period of about 15 minutes to about 360 minutes, about 30 minutes to about 240 minutes, or about 45 minutes to about 180 minutes (e.g., when measured with a USP disintegration apparatus in from any of about 500 ml to any of about 900 ml 0.1N HCL acidic media adjusted to pH with phosphate buffer solution, sodium hydroxide solution, or potassium hydroxide solution).

In certain embodiments, the shell composition does not dissolve at a pH of 2 at 15 minutes, 30 minutes, 45 minutes, 60 minutes, 90 minutes or 120 minutes (e.g., when measured with a USP Apparatus II with paddles at from any of about 50 RPM to any of about 250 RPM in from any of about 500 ml to any of about 900 ml 0.1N HCL acidic media adjusted to pH with phosphate buffer solution, sodium hydroxide solution, or potassium hydroxide solution).

In certain embodiments, the shell composition does not dissolve at a pH of 2 for a time period of at least about 15 minutes, at least about 30 minutes, at least about 45 minutes, at least about 60 minutes, at least about 90 minutes, or at least about 120 minutes (e.g., when measured with a USP Apparatus II with paddles at from any of about 50 RPM to any of about 250 RPM in from any of about 500 ml to any of about 900 ml 0.1N HCL acidic media adjusted to pH with phosphate buffer solution, sodium hydroxide solution, or potassium hydroxide solution).

In certain embodiments, the shell composition does not dissolve at a pH of 2 for a time period of about 15 minutes to about 360 minutes, about 30 minutes to about 240 minutes, or about 45 minutes to about 180 minutes (e.g., when measured with a USP Apparatus II with paddles at from any of about 50 RPM to any of about 250 RPM in from any of about 500 ml to any of about 900 ml 0.1N HCL acidic media adjusted to pH with phosphate buffer solution, sodium hydroxide solution, or potassium hydroxide solution).

In certain embodiments, the shell composition does not disintegrate at a pH of 2 at 15 minutes, 30 minutes, 45 minutes, 60 minutes, 90 minutes or 120 minutes (e.g., when measured with a USP disintegration apparatus in from any of about 500 ml to any of about 900 ml 0.1N HCL acidic media adjusted to pH with phosphate buffer solution, sodium hydroxide solution, or potassium hydroxide solution).

In certain embodiments, the shell composition does not disintegrate at a pH of 2 for a time period of at least about 15 minutes, at least about 30 minutes, at least about 45 minutes, at least about 60 minutes, at least about 90 minutes, or at least about 120 minutes (e.g., when measured with a USP disintegration apparatus in from any of about 500 ml to any of about 900 ml 0.1N HCL acidic media adjusted to pH with phosphate buffer solution, sodium hydroxide solution, or potassium hydroxide solution).

In certain embodiments, the shell composition does not disintegrate at a pH of 2 for a time period of about 15 minutes to about 360 minutes, about 30 minutes to about 240 minutes, or about 45 minutes to about 180 minutes (e.g., when measured with a USP disintegration apparatus in from any of about 500 ml to any of about 900 ml 0.1N HCL acidic media adjusted to pH with phosphate buffer solution, sodium hydroxide solution, or potassium hydroxide solution).

In certain embodiments, the shell composition does not dissolve at a pH of between 2 and 3 at 15 minutes, 30 minutes, 45 minutes, 60 minutes, 90 minutes or 120 minutes (e.g., when measured with a USP Apparatus II with paddles at from any of about 50 RPM to any of about 250 RPM in from any of about 500 ml to any of about 900 ml 0.1N HCL acidic media adjusted to pH with phosphate buffer solution, sodium hydroxide solution, or potassium hydroxide solution).

In certain embodiments, the shell composition does not dissolve at a pH of between 2 and 3 for a time period of at least about 15 minutes, at least about 30 minutes, at least about 45 minutes, at least about 60 minutes, at least about 90 minutes, or at least about 120 minutes (e.g., when measured with a USP Apparatus II with paddles at from any of about 50 RPM to any of about 250 RPM in from any of about 500 ml to any of about 900 ml 0.1N HCL acidic media adjusted to pH with phosphate buffer solution, sodium hydroxide solution, or potassium hydroxide solution).

In certain embodiments, the shell composition does not dissolve at a pH of between 2 and 3 for a time period of about 15 minutes to about 360 minutes, about 30 minutes to about 240 minutes, or about 45 minutes to about 180 minutes (e.g., when measured with a USP Apparatus II with paddles at from any of about 50 RPM to any of about 250 RPM in from any of about 500 ml to any of about 900 ml 0.1N HCL acidic media adjusted to pH with phosphate buffer solution, sodium hydroxide solution, or potassium hydroxide solution).

In certain embodiments, the shell composition does not disintegrate at a pH of between 2 and 3 at 15 minutes, 30 minutes, 45 minutes, 60 minutes, 90 minutes or 120 minutes (e.g., when measured with a USP disintegration apparatus in from any of about 500 ml to any of about 900 ml 0.1N HCL acidic media adjusted to pH with phosphate buffer solution, sodium hydroxide solution, or potassium hydroxide solution).

In certain embodiments, the shell composition does not disintegrate at a pH of between 2 and 3 for a time period of at least about 15 minutes, at least about 30 minutes, at least about 45 minutes, at least about 60 minutes, at least about 90 minutes, or at least about 120 minutes (e.g., when measured with a USP disintegration apparatus in from any of about 500 ml to any of about 900 ml 0.1N HCL acidic media adjusted to pH with phosphate buffer solution, sodium hydroxide solution, or potassium hydroxide solution).

In certain embodiments, the shell composition does not disintegrate at a pH of between 2 and 3 for a time period of about 15 minutes to about 360 minutes, about 30 minutes to about 240 minutes, or about 45 minutes to about 180 minutes (e.g., when measured with a USP disintegration apparatus in from any of about 500 ml to any of about 900 ml 0.1N HCL acidic media adjusted to pH with phosphate buffer solution, sodium hydroxide solution, or potassium hydroxide solution).

In certain embodiments, the shell composition does not dissolve at a pH of 3 at 15 minutes, 30 minutes, 45 minutes, 60 minutes, 90 minutes or 120 minutes (e.g., when measured with a USP Apparatus II with paddles at from any of about 50 RPM to any of about 250 RPM in from any of about 500 ml to any of about 900 ml 0.1N HCL acidic media adjusted to pH with phosphate buffer solution, sodium hydroxide solution, or potassium hydroxide solution).

In certain embodiments, the shell composition does not dissolve at a pH of 3 for a time period of at least about 15 minutes, at least about 30 minutes, at least about 45 minutes, at least about 60 minutes, at least about 90 minutes, or at least about 120 minutes (e.g., when measured with a USP Apparatus II with paddles at from any of about 50 RPM to any of about 250 RPM in from any of about 500 ml to any of about 900 ml 0.1N HCL acidic media adjusted to pH with phosphate buffer solution, sodium hydroxide solution, or potassium hydroxide solution).

In certain embodiments, the shell composition does not dissolve at a pH of 3 for a time period of about 15 minutes to about 360 minutes, about 30 minutes to about 240 minutes, or about 45 minutes to about 180 minutes (e.g., when measured with a USP Apparatus II with paddles at from any of about 50 RPM to any of about 250 RPM in from any of about 500 ml to any of about 900 ml 0.1N HCL acidic media adjusted to pH with phosphate buffer solution, sodium hydroxide solution, or potassium hydroxide solution).

In certain embodiments, the shell composition does not disintegrate at a pH of 3 at 15 minutes, 30 minutes, 45 minutes, 60 minutes, 90 minutes or 120 minutes (e.g., when measured with a USP disintegration apparatus in from any of about 500 ml to any of about 900 ml 0.1N HCL acidic media adjusted to pH with phosphate buffer solution, sodium hydroxide solution, or potassium hydroxide solution).

In certain embodiments, the shell composition does not disintegrate at a pH of 3 for a time period of at least about 15 minutes, at least about 30 minutes, at least about 45 minutes, at least about 60 minutes, at least about 90 minutes, or at least about 120 minutes (e.g., when measured with a USP disintegration apparatus in from any of about 500 ml to any of about 900 ml 0.1N HCL acidic media adjusted to pH with phosphate buffer solution, sodium hydroxide solution, or potassium hydroxide solution).

In certain embodiments, the shell composition does not disintegrate at a pH of 3 for a time period of about 15 minutes to about 360 minutes, about 30 minutes to about 240 minutes, or about 45 minutes to about 180 minutes (e.g., when measured with a USP disintegration apparatus in from any of about 500 ml to any of about 900 ml 0.1N HCL acidic media adjusted to pH with phosphate buffer solution, sodium hydroxide solution, or potassium hydroxide solution).

In certain embodiments, the shell composition does not dissolve at a pH of between 3 and 4 at 15 minutes, 30 minutes, 45 minutes, 60 minutes, 90 minutes or 120 minutes (e.g., when measured with a USP Apparatus II with paddles at from any of about 50 RPM to any of about 250 RPM in from any of about 500 ml to any of about 900 ml 0.1N HCL acidic media adjusted to pH with phosphate buffer solution, sodium hydroxide solution, or potassium hydroxide solution).

In certain embodiments, the shell composition does not dissolve at a pH of 1.2 for a time period of at least about 15 minutes, at least about 30 minutes, at least about 45 minutes, at least about 60 minutes, at least about 90 minutes, or at least about 120 minutes (e.g., when measured with a USP Apparatus II with paddles at from any of about 50 RPM to any of about 250 RPM in from any of about 500 ml to any of about 900 ml 0.1N HCL acidic media adjusted to pH with phosphate buffer solution, sodium hydroxide solution, or potassium hydroxide solution).

In certain embodiments, the shell composition does not dissolve at a pH of between 3 and 4 for a time period of about 15 minutes to about 360 minutes, about 30 minutes to about 240 minutes, or about 45 minutes to about 180 minutes (e.g., when measured with a USP Apparatus II with paddles at from any of about 50 RPM to any of about 250 RPM in from any of about 500 ml to any of about 900 ml 0.1N HCL acidic media adjusted to pH with phosphate buffer solution, sodium hydroxide solution, or potassium hydroxide solution).

In certain embodiments, the shell composition does not disintegrate at a pH of between 3 and 4 at 15 minutes, 30 minutes, 45 minutes, 60 minutes, 90 minutes or 120 minutes (e.g., when measured with a USP disintegration apparatus in from any of about 500 ml to any of about 900 ml 0.1N HCL acidic media adjusted to pH with phosphate buffer solution, sodium hydroxide solution, or potassium hydroxide solution).

In certain embodiments, the shell composition does not disintegrate at a pH of between 3 and 4 for a time period of at least about 15 minutes, at least about 30 minutes, at least about 45 minutes, at least about 60 minutes, at least about 90 minutes, or at least about 120 minutes (e.g., when measured with a USP disintegration apparatus in from any of about 500 ml to any of about 900 ml 0.1N HCL acidic media adjusted to pH with phosphate buffer solution, sodium hydroxide solution, or potassium hydroxide solution).

In certain embodiments, the shell composition does not disintegrate at a pH of between 3 and 4 for a time period of about 15 minutes to about 360 minutes, about 30 minutes to about 240 minutes, or about 45 minutes to about 180 minutes (e.g., when measured with a USP disintegration apparatus in from any of about 500 ml to any of about 900 ml 0.1N HCL acidic media adjusted to pH with phosphate buffer solution, sodium hydroxide solution, or potassium hydroxide solution).

In certain embodiments, the shell composition does not dissolve at a pH of 4 at 15 minutes, 30 minutes, 45 minutes, 60 minutes, 90 minutes or 120 minutes (e.g., when measured with a USP Apparatus II with paddles at from any of about 50 RPM to any of about 250 RPM in from any of about 500 ml to any of about 900 ml 0.1N HCL acidic media adjusted to pH with phosphate buffer solution, sodium hydroxide solution, or potassium hydroxide solution).

In certain embodiments, the shell composition does not dissolve at a pH of 4 for a time period of at least about 15 minutes, at least about 30 minutes, at least about 45 minutes, at least about 60 minutes, at least about 90 minutes, or at least about 120 minutes (e.g., when measured with a USP Apparatus II with paddles at from any of about 50 RPM to any of about 250 RPM in from any of about 500 ml to any of about 900 ml 0.1N HCL acidic media adjusted to pH with phosphate buffer solution, sodium hydroxide solution, or potassium hydroxide solution).

In certain embodiments, the shell composition does not dissolve at a pH of 4 for a time period of about 15 minutes to about 360 minutes, about 30 minutes to about 240 minutes, or about 45 minutes to about 180 minutes (e.g., when measured with a USP Apparatus II with paddles at from any of about 50 RPM to any of about 250 RPM in from any of about 500 ml to any of about 900 ml 0.1N HCL acidic media adjusted to pH with phosphate buffer solution, sodium hydroxide solution, or potassium hydroxide solution).

In certain embodiments, the shell composition does not disintegrate at a pH of 4 at 15 minutes, 30 minutes, 45 minutes, 60 minutes, 90 minutes or 120 minutes (e.g., when measured with a USP disintegration apparatus in from any of about 500 ml to any of about 900 ml 0.1N HCL acidic media adjusted to pH with phosphate buffer solution, sodium hydroxide solution, or potassium hydroxide solution).

In certain embodiments, the shell composition does not disintegrate at a pH of 4 for a time period of at least about 15 minutes, at least about 30 minutes, at least about 45 minutes, at least about 60 minutes, at least about 90 minutes, or at least about 120 minutes (e.g., when measured with a USP disintegration apparatus in from any of about 500 ml to any of about 900 ml 0.1N HCL acidic media adjusted to pH with phosphate buffer solution, sodium hydroxide solution, or potassium hydroxide solution).

In certain embodiments, the shell composition does not disintegrate at a pH of 4 for a time period of about 15 minutes to about 360 minutes, about 30 minutes to about 240 minutes, or about 45 minutes to about 180 minutes (e.g., when measured with a USP disintegration apparatus in from any of about 500 ml to any of about 900 ml 0.1N HCL acidic media adjusted to pH with phosphate buffer solution, sodium hydroxide solution, or potassium hydroxide solution).

In certain embodiments, the shell composition does not dissolve at a pH of between 4 and 5 at 15 minutes, 30 minutes, 45 minutes, 60 minutes, 90 minutes or 120 minutes (e.g., when measured with a USP Apparatus II with paddles at from any of about 50 RPM to any of about 250 RPM in from any of about 500 ml to any of about 900 ml 0.1N HCL acidic media adjusted to pH with phosphate buffer solution, sodium hydroxide solution, or potassium hydroxide solution).

In certain embodiments, the shell composition does not dissolve at a pH of between 4 and 5 for a time period of at least about 15 minutes, at least about 30 minutes, at least about 45 minutes, at least about 60 minutes, at least about 90 minutes, or at least about 120 minutes (e.g., when measured with a USP Apparatus II with paddles at from any of about 50 RPM to any of about 250 RPM in from any of about 500 ml to any of about 900 ml 0.1N HCL acidic media adjusted to pH with phosphate buffer solution, sodium hydroxide solution, or potassium hydroxide solution).

In certain embodiments, the shell composition does not dissolve at a pH of between 4 and 5 for a time period of about 15 minutes to about 360 minutes, about 30 minutes to about 240 minutes, or about 45 minutes to about 180 minutes (e.g., when measured with a USP Apparatus II with paddles at from any of about 50 RPM to any of about 250 RPM in from any of about 500 ml to any of about 900 ml 0.1N HCL acidic media adjusted to pH with phosphate buffer solution, sodium hydroxide solution, or potassium hydroxide solution).

In certain embodiments, the shell composition does not disintegrate at a pH of between 4 and 5 at 15 minutes, 30 minutes, 45 minutes, 60 minutes, 90 minutes or 120 minutes (e.g., when measured with a USP disintegration apparatus in from any of about 500 ml to any of about 900 ml 0.1N HCL acidic media adjusted to pH with phosphate buffer solution, sodium hydroxide solution, or potassium hydroxide solution).

In certain embodiments, the shell composition does not disintegrate at a pH of between 4 and 5 for a time period of at least about 15 minutes, at least about 30 minutes, at least about 45 minutes, at least about 60 minutes, at least about 90 minutes, or at least about 120 minutes (e.g., when measured with a USP disintegration apparatus in from any of about 500 ml to any of about 900 ml 0.1N HCL acidic media adjusted to pH with phosphate buffer solution, sodium hydroxide solution, or potassium hydroxide solution).

In certain embodiments, the shell composition does not disintegrate at a pH of between 4 and 5 for a time period of about 15 minutes to about 360 minutes, about 30 minutes to about 240 minutes, or about 45 minutes to about 180 minutes (e.g., when measured with a USP disintegration apparatus in from any of about 500 ml to any of about 900 ml 0.1N HCL acidic media adjusted to pH with phosphate buffer solution, sodium hydroxide solution, or potassium hydroxide solution).

In certain embodiments, the shell composition does not dissolve at a pH of 5 at 15 minutes, 30 minutes, 45 minutes, 60 minutes, 90 minutes or 120 minutes (e.g., when measured with a USP Apparatus II with paddles at from any of about 50 RPM to any of about 250 RPM in from any of about 500 ml to any of about 900 ml 0.1N HCL acidic media adjusted to pH with phosphate buffer solution, sodium hydroxide solution, or potassium hydroxide solution).

In certain embodiments, the shell composition does not dissolve at a pH of 5 for a time period of at least about 15 minutes, at least about 30 minutes, at least about 45 minutes, at least about 60 minutes, at least about 90 minutes, or at least about 120 minutes (e.g., when measured with a USP Apparatus II with paddles at from any of about 50 RPM to any of about 250 RPM in from any of about 500 ml to any of about 900 ml 0.1N HCL acidic media adjusted to pH with phosphate buffer solution, sodium hydroxide solution, or potassium hydroxide solution).

In certain embodiments, the shell composition does not dissolve at a pH of 5 for a time period of about 15 minutes to about 360 minutes, about 30 minutes to about 240 minutes, or about 45 minutes to about 180 minutes (e.g., when measured with a USP Apparatus II with paddles at from any of about 50 RPM to any of about 250 RPM in from any of about 500 ml to any of about 900 ml 0.1N HCL acidic media adjusted to pH with phosphate buffer solution, sodium hydroxide solution, or potassium hydroxide solution).

In certain embodiments, the shell composition does not disintegrate at a pH of 5 at 15 minutes, 30 minutes, 45 minutes, 60 minutes, 90 minutes or 120 minutes (e.g., when measured with a USP disintegration apparatus in from any of about 500 ml to any of about 900 ml 0.1N HCL acidic media adjusted to pH with phosphate buffer solution, sodium hydroxide solution, or potassium hydroxide solution).

In certain embodiments, the shell composition does not disintegrate at a pH of 5 for a time period of at least about 15 minutes, at least about 30 minutes, at least about 45 minutes, at least about 60 minutes, at least about 90 minutes, or at least about 120 minutes (e.g., when measured with a USP disintegration apparatus in from any of about 500 ml to any of about 900 ml 0.1N HCL acidic media adjusted to pH with phosphate buffer solution, sodium hydroxide solution, or potassium hydroxide solution).

In certain embodiments, the shell composition does not disintegrate at a pH of 5 for a time period of about 15 minutes to about 360 minutes, about 30 minutes to about 240 minutes, or about 45 minutes to about 180 minutes (e.g., when measured with a USP disintegration apparatus in from any of about 500 ml to any of about 900 ml 0.1N HCL acidic media adjusted to pH with phosphate buffer solution, sodium hydroxide solution, or potassium hydroxide solution).

In certain embodiments, the shell composition does not dissolve at a pH of between 5 and 6 at 15 minutes, 30 minutes, 45 minutes, 60 minutes, 90 minutes or 120 minutes (e.g., when measured with a USP Apparatus II with paddles at from any of about 50 RPM to any of about 250 RPM in from any of about 500 ml to any of about 900 ml 0.1N HCL acidic media adjusted to pH with phosphate buffer solution, sodium hydroxide solution, or potassium hydroxide solution).

In certain embodiments, the shell composition does not dissolve at a pH of between 5 and 6 for a time period of at least about 15 minutes, at least about 30 minutes, at least about 45 minutes, at least about 60 minutes, at least about 90 minutes, or at least about 120 minutes (e.g., when measured with a USP Apparatus II with paddles at from any of about 50 RPM to any of about 250 RPM in from any of about 500 ml to any of about 900 ml 0.1N HCL acidic media adjusted to pH with phosphate buffer solution, sodium hydroxide solution, or potassium hydroxide solution).

In certain embodiments, the shell composition does not dissolve at a pH of between 5 and 6 for a time period of about 15 minutes to about 360 minutes, about 30 minutes to about 240 minutes, or about 45 minutes to about 180 minutes (e.g., when measured with a USP Apparatus II with paddles at from any of about 50 RPM to any of about 250 RPM in from any of about 500 ml to any of about 900 ml 0.1N HCL acidic media adjusted to pH with phosphate buffer solution, sodium hydroxide solution, or potassium hydroxide solution).

In certain embodiments, the shell composition does not disintegrate at a pH of between 5 and 6 at 15 minutes, 30 minutes, 45 minutes, 60 minutes, 90 minutes or 120 minutes (e.g., when measured with a USP disintegration apparatus in from any of about 500 ml to any of about 900 ml 0.1N HCL acidic media adjusted to pH with phosphate buffer solution, sodium hydroxide solution, or potassium hydroxide solution).

In certain embodiments, the shell composition does not disintegrate at a pH of between 5 and 6 for a time period of at least about 15 minutes, at least about 30 minutes, at least about 45 minutes, at least about 60 minutes, at least about 90 minutes, or at least about 120 minutes (e.g., when measured with a USP disintegration apparatus in from any of about 500 ml to any of about 900 ml 0.1N HCL acidic media adjusted to pH with phosphate buffer solution, sodium hydroxide solution, or potassium hydroxide solution).

In certain embodiments, the shell composition does not disintegrate at a pH of between 5 and 6 for a time period of about 15 minutes to about 360 minutes, about 30 minutes to about 240 minutes, or about 45 minutes to about 180 minutes (e.g., when measured with a USP disintegration apparatus in from any of about 500 ml to any of about 900 ml 0.1N HCL acidic media adjusted to pH with phosphate buffer solution, sodium hydroxide solution, or potassium hydroxide solution).

In certain embodiments, the shell composition does not dissolve at a pH of 6 at 15 minutes, 30 minutes, 45 minutes, 60 minutes, 90 minutes or 120 minutes (e.g., when measured with a USP Apparatus II with paddles at from any of about 50 RPM to any of about 250 RPM in from any of about 500 ml to any of about 900 ml 0.1N HCL acidic media adjusted to pH with phosphate buffer solution, sodium hydroxide solution, or potassium hydroxide solution).

In certain embodiments, the shell composition does not dissolve at a pH of 6 for a time period of at least about 15 minutes, at least about 30 minutes, at least about 45 minutes, at least about 60 minutes, at least about 90 minutes, or at least about 120 minutes (e.g., when measured with a USP Apparatus II with paddles at from any of about 50 RPM to any of about 250 RPM in from any of about 500 ml to any of about 900 ml 0.1N HCL acidic media adjusted to pH with phosphate buffer solution, sodium hydroxide solution, or potassium hydroxide solution).

In certain embodiments, the shell composition does not dissolve at a pH of 6 for a time period of about 15 minutes to about 360 minutes, about 30 minutes to about 240 minutes, or about 45 minutes to about 180 minutes (e.g., when measured with a USP Apparatus II with paddles at from any of about 50 RPM to any of about 250 RPM in from any of about 500 ml to any of about 900 ml 0.1N HCL acidic media adjusted to pH with phosphate buffer solution, sodium hydroxide solution, or potassium hydroxide solution).

In certain embodiments, the shell composition does not disintegrate at a pH of 6 at 15 minutes, 30 minutes, 45 minutes, 60 minutes, 90 minutes or 120 minutes (e.g., when measured with a USP disintegration apparatus in from any of about 500 ml to any of about 900 ml 0.1N HCL acidic media adjusted to pH with phosphate buffer solution, sodium hydroxide solution, or potassium hydroxide solution).

In certain embodiments, the shell composition does not disintegrate at a pH of 6 for a time period of at least about 15 minutes, at least about 30 minutes, at least about 45 minutes, at least about 60 minutes, at least about 90 minutes, or at least about 120 minutes (e.g., when measured with a USP disintegration apparatus in from any of about 500 ml to any of about 900 ml 0.1N HCL acidic media adjusted to pH with phosphate buffer solution, sodium hydroxide solution, or potassium hydroxide solution).

In certain embodiments, the shell composition does not disintegrate at a pH of 6 for a time period of about 15 minutes to about 360 minutes, about 30 minutes to about 240 minutes, or about 45 minutes to about 180 minutes (e.g., when measured with a USP disintegration apparatus in from any of about 500 ml to any of about 900 ml 0.1N HCL acidic media adjusted to pH with phosphate buffer solution, sodium hydroxide solution, or potassium hydroxide solution).

In certain embodiments, the shell composition does not dissolve at a pH of less than 8.4, less than 8.3, less than 8.2, less than 8.1, less than 8.0, less than 7.9, less than 7.8, less than 7.7, less than 7.6, less than 7.5, less than 7.4, less than 7.3, less than 7.2, less than 7.1, less than 7.0, less than 6.9, less than 6.8, less than 6.7, less than 6.6, less than 6.5, less than 6.4, less than 6.3, less than 6.2, less than 6.1, less than 6.0, less than 5.9, less than 5.8, less than 5.7, less than 5.6, less than 5.5, less than 5.4, less than 5.3, less than 5.2, less than 5.1, less than 5.0, less than 4.9, less than 4.8, less than 4.7, less than 4.6, less than 4.5, less than 4.4, less than 4.3, less than 4.2, less than 4.1, less than 4.0, less than 3.9, less than 3.8, less than 3.7, less than 3.6, less than 3.5, less than 3.4, less than 3.3, less than 3.2, less than 3.1, less than 3.0, less than 2.9, less than 2.8, less than 2.7, less than 2.6, less than 2.5, less than 2.4, less than 2.3, less than 2.2, less than 2.1, less than 2.0, less than 1.9, less than 1.8, less than 1.7, less than 1.6, less than 1.5, less than 1.4, less than 1.3 or less than 1.2 for a time period of at least about 15 minutes, at least about 30 minutes, at least about 45 minutes, at least about 60 minutes, at least about 90 minutes, or at least about 120 minutes (e.g., when measured with a USP Apparatus II with paddles at from any of about 50 RPM to any of about 250 RPM in from any of about 500 ml to any of about 900 ml 0.1N HCL acidic media adjusted to pH with phosphate buffer solution, sodium hydroxide solution, or potassium hydroxide solution).

In certain embodiments, the shell composition does not dissolve at a pH of less than 8.4, less than 8.3, less than 8.2, less than 8.1, less than 8.0, less than 7.9, less than 7.8, less than 7.7, less than 7.6, less than 7.5, less than 7.4, less than 7.3, less than 7.2, less than 7.1, less than 7.0, less than 6.9, less than 6.8, less than 6.7, less than 6.6, less than 6.5, less than 6.4, less than 6.3, less than 6.2, less than 6.1, less than 6.0, less than 5.9, less than 5.8, less than 5.7, less than 5.6, less than 5.5, less than 5.4, less than 5.3, less than 5.2, less than 5.1, less than 5.0, less than 4.9, less than 4.8, less than 4.7, less than 4.6, less than 4.5, less than 4.4, less than 4.3, less than 4.2, less than 4.1, less than 4.0, less than 3.9, less than 3.8, less than 3.7, less than 3.6, less than 3.5, less than 3.4, less than 3.3, less than 3.2, less than 3.1, less than 3.0, less than 2.9, less than 2.8, less than 2.7, less than 2.6, less than 2.5, less than 2.4, less than 2.3, less than 2.2, less than 2.1, less than 2.0, less than 1.9, less than 1.8, less than 1.7, less than 1.6, less than 1.5, less than 1.4, less than 1.3, or less than 1.2 for a time period of about 15 minutes to about 360 minutes, about 30 minutes to about 240 minutes, or about 45 minutes to about 180 minutes (e.g., when measured with a USP Apparatus II with paddles at from any of about 50 RPM to any of about 250 RPM in from any of about 500 ml to any of about 900 ml 0.1N HCL acidic media adjusted to pH with phosphate buffer solution, sodium hydroxide solution, or potassium hydroxide solution).

In certain embodiments, the shell composition does not disintegrate at a pH of less than 8.4, less than 8.3, less than 8.2, less than 8.1, less than 8.0, less than 7.9, less than 7.8, less than 7.7, less than 7.6, less than 7.5, less than 7.4, less than 7.3, less than 7.2, less than 7.1, less than 7.0, less than 6.9, less than 6.8, less than 6.7, less than 6.6, less than 6.5, less than 6.4, less than 6.3, less than 6.2, less than 6.1, less than 6.0, less than 5.9, less than 5.8, less than 5.7, less than 5.6, less than 5.5, less than 5.4, less than 5.3, less than 5.2, less than 5.1, less than 5.0, less than 4.9, less than 4.8, less than 4.7, less than 4.6, less than 4.5, less than 4.4, less than 4.3, less than 4.2, less than 4.1, less than 4.0, less than 3.9, less than 3.8, less than 3.7, less than 3.6, less than 3.5, less than 3.4, less than 3.3, less than 3.2, less than 3.1, less than 3.0, less than 2.9, less than 2.8, less than 2.7, less than 2.6, less than 2.5, less than 2.4, less than 2.3, less than 2.2, less than 2.1, less than 2.0, less than 1.9, less than 1.8, less than 1.7, less than 1.6, less than 1.5, less than 1.4, less than 1.3 or less than 1.2 for a time period of at least about 15 minutes, at least about 30 minutes, at least about 45 minutes, at least about 60 minutes, at least about 90 minutes, or at least about 120 minutes (e.g., when measured with a USP disintegration apparatus in from any of about 500 ml to any of about 900 ml 0.1N HCL acidic media adjusted to pH with phosphate buffer solution, sodium hydroxide solution, or potassium hydroxide solution).

In certain embodiments, the shell composition does not disintegrate at a pH of less than 8.4, less than 8.3, less than 8.2, less than 8.1, less than 8.0, less than 7.9, less than 7.8, less than 7.7, less than 7.6, less than 7.5, less than 7.4, less than 7.3, less than 7.2, less than 7.1, less than 7.0, less than 6.9, less than 6.8, less than 6.7, less than 6.6, less than 6.5, less than 6.4, less than 6.3, less than 6.2, less than 6.1, less than 6.0, less than 5.9, less than 5.8, less than 5.7, less than 5.6, less than 5.5, less than 5.4, less than 5.3, less than 5.2, less than 5.1, less than 5.0, less than 4.9, less than 4.8, less than 4.7, less than 4.6, less than 4.5, less than 4.4, less than 4.3, less than 4.2, less than 4.1, less than 4.0, less than 3.9, less than 3.8, less than 3.7, less than 3.6, less than 3.5, less than 3.4, less than 3.3, less than 3.2, less than 3.1, less than 3.0, less than 2.9, less than 2.8, less than 2.7, less than 2.6, less than 2.5, less than 2.4, less than 2.3, less than 2.2, less than 2.1, less than 2.0, less than 1.9, less than 1.8, less than 1.7, less than 1.6, less than 1.5, less than 1.4, less than 1.3 or less than 1.2 for a time period of about 15 minutes to about 360 minutes, about 30 minutes to about 240 minutes, or about 45 minutes to about 180 minutes (e.g., when measured with a USP disintegration apparatus in from any of about 500 ml to any of about 900 ml 0.1N HCL acidic media adjusted to pH with phosphate buffer solution, sodium hydroxide solution, or potassium hydroxide solution).

By virtue of the present invention, the pH that is suitable to dissolve and/or disintegrate and/or rupture the shell composition and release the fill material can be selected in order to program the release of the active agent to inhibit premature release of the active agent in acidic portions of the gastrointestinal tract (e.g., gastric environment where the pH is between 1.2 and 3.5) and instead to release the active agent at the intended portion of the gastro-intestinal tract. For example, the duodenum has a typical pH ranging from 7.0 to 8.5; the small and large intestine typically have a pH of 4.0 to 7.0; the colon has a typical pH of 6.5 and the jejunum has a typical pH of 6.1 to 7.2. In one embodiment, the shell composition may be adjusted to target release of the active agent in the duodenum at a pH of about 7.0 to about 8.5. In one embodiment, the shell composition may be adjusted to target release of the active agent in the small and large intestine at a pH of about 4.0 to about 7.0. In one embodiment, the shell composition may be adjusted to target release of the active agent in the colon at a pH of about 6.5. In one embodiment, the shell composition may be adjusted to target release of the active agent in the jejunum at a pH of about 6.1 to about 7.2.

In certain embodiments, the combination of pectin and methyl acrylic copolymer in the pH dependent shell compositions raises the rupture threshold of the capsule to pH 7.5 to 8.5, providing means to deliver active agent into the small intestine.

### Method of Preparation

Encapsulation of the fill material can be accomplished in any conventional manner. As an example, a rotary die encapsulation may be used.

According to an embodiment, a pH dependent softgel capsule is prepared by the process comprising the steps of: (a) preparing the fill material, said fill material comprising at least one active agent; and (b) encapsulating the fill material of step (a) in a pH dependent shell composition. The encapsulation process according to step (b) may further comprise a sub-step of preparing the pH dependent shell composition by, for example, admixing a gelatin, dextrose, a pectin, a synthetic polymer, optionally a plasticizer, and optionally a stabilizer/binder. In an embodiment, the sub-step of preparing the pH dependent shell composition includes, for example, admixing a gelatin, dextrose, a pectin, an organic acid, optionally a plasticizer, and optionally a stabilizer/binder. In an embodiment, the sub-step of preparing the pH dependent shell composition includes, for example, admixing a gelatin, dextrose, a pectin, a synthetic polymer, an organic acid, optionally a plasticizer, and optionally a stabilizer/binder.

The ribbon thickness of the pH dependent shell composition (as used for example during rotary die encapsulation) may also be tuned to control the pH dependent dissolution profile of the final pH dependent softgel capsule. The ribbon thickness of the pH dependent shell composition may range, without limitations, from any of about 0.02 inches, about 0.022 inches, about 0.024 inches, about 0.026 inches, about 0.028 inches, or about 0.030 inches to any of about 0.032 inches, about 0.034 inches, about 0.036 inches, about 0.038 inches, about 0.04 inches, about 0.042 inches, about 0.044 inches, or about 0.050 inches or any sub-range or single value therein.

In certain embodiments, the pH dependent softgel capsule (e.g., after encapsulation) may be dried and optionally cured. Curing the softgel capsule may be performed at a temperature ranging from about 25° C. to about 75° C., about 25° C. to about 70° C., from about 30° C. to about 60° C., or from about 35° C. to 50° C. The curing temperature should be high enough to enhance the delayed release properties of the softgel capsules but not so high that it would melt the softgel capsule.

The duration of curing may range from about 12 hours to about 168 hours, from about 18 hours to about 120 hours, from about 24 hours to about 72 hours, about 24 hours, about 48 hours, about 72 hours, or any sub-range or single values therein. In an embodiment, the curing of the softgel capsule may be performed at a temperature of about 40° C. for about 24 hours. In an embodiment, the curing of the softgel capsule may be performed at a temperature of about 40° C. for about 48 hours. In an embodiment, the curing of the softgel capsule may be performed at a temperature of about 40° C. for about 72 hours. In certain embodiments, the curing may occur in air (without any particular controls as to the content of nitrogen or oxygen or humidity). In certain embodiments, the curing may occur under inert conditions (e.g., in nitrogen).

In an embodiment, the process for preparing a pH dependent softgel capsule comprises, consists essentially of, or consists of a) preparing any of the fill materials described herein; b) encapsulating the fill material from step a) in any of the pH dependent shell compositions described herein (e.g., via rotary die encapsulation); c) drying the encapsulated pH dependent softgel capsules (e.g., by tumble drying or regular drying in a basket without tumbling); and optionally d) curing the pH dependent softgel capsule in accordance with any of the curing conditions described herein.

In certain embodiments, drying is performed at about 10° C. to about 50° C., about 15° C. to about 40° C., or about 20° C. to about 35° C. at a relative humidity of about 5% to about 40%, about 10% to about 30%, or about 15% to about 25%.

In certain embodiments, reference to drying and curing should be distinguished here. The purpose of drying the delayed release softgel capsules described herein is to remove excess water from the delayed release softgel capsule immediately after encapsulation. So, the capsules will be physically stable. The purpose of curing the delayed release softgel capsules described herein is to enhance the delayed release property of the delayed release softgel capsule. Hence, the presence of a drying step is not the same as a curing step and similarly the presence of a curing step is not the same as a drying step.

In certain embodiments, the pH dependent shell compositions described herein exhibit any of the delayed release properties described herein (e.g., in accordance with any of the dissolution or disintegration profiles described herein) without being cured. For instance, in certain embodiments, the inclusion of the synthetic polymer may enhance the delayed release properties of the softgel capsule without needing to further cure the softgel capsule.

In certain embodiments, the process for preparing the softgel capsules described herein may further include washing the softgel capsule with an organic acid. Suitable organic acids include, without limitations, lactic acid, tannic acid, citric acid, acetic acid, or a combination thereof. In certain embodiments, washing the softgel capsule with an organic acid further enhances the robustness of the softgel capsule and its delayed release properties (as evidenced by achieving, e.g., any one or more of the dissolution or disintegration release profiled described herein).

### Softgel Capsule Stability

In certain embodiments, delayed release softgel capsules having the pH dependent shell compositions described herein are chemically and physically stable.

For instance, their chemical stability may be evidenced by the content of the active agent in the fill material (e.g., content of fish oil constituents when the fill material includes fish oil). In certain embodiments, the content of the fill material constituents is substantially similar (or within specifications), after storage for up to 12 months, up to 6 months, up to 3 months, or up to 1 months (at ambient conditions or at stressed conditions of 40° C. and 75% relative humidity for any of these durations) as compared to the raw material before storage for said duration.

In certain embodiments, the physical stability of the delayed release softgel capsules may be evidenced by the dissolution profile of the capsule in acidic medium and in buffer medium. For instance, the dissolution profile of the capsule in acidic medium and in buffer medium is substantially similar (or within specifications), after storage for up to 12 months, up to 6 months, up to 3 months, or up to 1 months (at ambient conditions or at stressed conditions of 40° C. and 75% relative humidity for any of these durations) as compared to the dissolution profile of the capsule before storage.

The term “substantially similar” may refer to a particular value being within about 30%, within about 25%, within about 20%, within about 15%, within about 10%, within about 5%, or within about 1% of a corresponding comparative value. The percentage being calculated based on the face value of the comparative value. For instance, a dissolution time range of 27 minutes to 33 minutes may be considered within 10% of comparative dissolution time of 30 minutes.

In certain embodiments, the pH dependent shell composition described herein produce a robust delayed release softgel capsule that has little or no premature release of the fill material in acidic environment (e.g., stomach environment). For instance, delayed release softgel capsules described herein may release up to about 10 wt %, up to about 9 wt %, up to about 8 wt %, up to about 7 wt %, up to about 6 wt %, up to about 5 wt %, up to about 4 wt %, up to about 3 wt %, up to about 1 wt %, or 0 wt %, of the fill material based on total weight of the fill material in acid stage after exposure to the acid stage (e.g., as defined for the dissolution tests or disintegration tests described herein) for up to about 120 minutes, up to about 105 minutes, up to about 90 minutes, up to about 75 minutes, up to about 60 minutes, up to about 45 minutes, up to about 30 minutes, up to about 15 minutes, up to about 10 minutes, or up to about 5 minutes.

### EXAMPLES

Specific embodiments of the invention will now be demonstrated by reference to the following examples. It should be understood that these examples are disclosed solely by way of illustrating the invention and should not be taken in any way to limit the scope of the present invention.

### Example 1—Addition of Synthetic Polymer Sealant to Wet Gel Mass to Inhibit Premature Release in Acidic Stage

A pH dependent shell composition having the dry shell composition of Table 1 was prepared.

TABLE 1
Dry Shell Composition of a pH Dependent Shell Composition Including a
Synthetic Polymer (Compositions of Lots 20MC-59A and 20MC-59B)

| | wt % (based on total weight |
|---|---|
| INGREDIENT | of dry shell composition) |
| Gelatin | 40-65 |
| Glycerin | 25-55 |
| Pectin | 6-15 |
| Synthetic Polymer (Kollicoat MAE-100P) | 0.5-10 |
| Gellan gum | 0.1-2 |
| Dextrose | 0.01-0.5 |
| Water | 6-15 |
| Total | 100 |

The synthetic polymer used in this example, Kollicoat MAE-100P, a methacrylic aid-ethyl acrylate copolymer (1:1), functioned as a sealant to stop the seepage of fill materials from capsule seals.

Fish oil and polyethylene glycol 400 were encapsulated into pH dependent shell compositions having the dry shell composition of Table 1 and dried. After drying, the softgel capsules were subjected to two-stage dissolution test conducted on a USP Apparatus II with a paddle at 50 RPM, where in the first stage, the softgel capsules were in acid stage (0.1N HCl) for two hours (120 minutes), and in the second stage, the softgel capsules were in buffer stage (Buffer pH 6.8). The results are summarized in Table 2.

TABLE 2
Two Stage Dissolution Test Result on Softgel Capsules Having pH
Dependent Shell Composition of Table 1

| | | Dissolution T0 @50 RPM | |
|---|---|---|---|
| Lot No | Fill | 0.1N HCl | Buffer pH 6.8 |
| 20MC-59A | Fish Oil | Intact for 120 mins | Ruptured in 4 |
| | | (No premature | minutes |
| | | releases) | |
| 20MC-59B | Polyethylene glycol | Intact for 120 mins | Ruptured in 8 |
| | 400 | (No premature | minutes |
| | | releases) | |

The fish oil softgel capsules (Lot No. 20MC-59A), before aging (T0) and after aging for 3 months at 40° C. and 75% relative humidity (T3), were also subjected to two stage dissolution with paddle speed of 100 RPM (all other dissolution test conditions being the same as for the results depicted in Table 2). The softgel capsules also stayed intact for 120 minutes in 0.1N HCl and ruptured in pH 6.8 buffer (Table 3).

TABLE 3
Two Stage Dissolution Test Result on Softgel Capsules Having pH
Dependent Shell Composition of Table 1 with Fish Oil (Lot 20MC-59A)

| Lot No | Fill | 0.1N HCl | Buffer pH 6.8 |
|---|---|---|---|
| 20MC-59A | Fish Oil | Dissolution T0 @100 RPM | |
| | | Intact for 120 mins | Ruptured in 4 |
| | | (No premature | minutes |
| | | releases) | |
| | | Dissolution T3 (40° C./75% RH) @100 | |
| | | RPM | |
| | | Intact for 120 mins | Ruptured in 8 |
| | | (No premature | minutes |
| | | releases) | |

Fish oil softgel capsules (Lot No. 20MC-59A), after aging for 3 months at 40° C. and 75% relative humidity (T3), were also subjected to a two-stage disintegration test using a USP disintegration apparatus, where in the first stage, the softgel capsules were in acid stage (0.1N HCl) for one hour (60 minutes), and in the second stage, the softgel capsules were in buffer stage (Buffer pH 6.8). In the disintegration test, the capsules stayed intact for 60 minutes, and ruptured in pH 6.8 buffer in 5 minutes (Table 4).

TABLE 4
Two Stage Disintegration Test Result on Softgel Capsules Having pH
Dependent Shell Composition of Table 1 with Fish Oil Fill (Lot 20MC-
59A), After Aging for 3 Months at 40° C./75% RH (T3)

| | | Disintegration | |
|---|---|---|---|
| Lot No | Fill | 0.1N HCl | Buffer pH 6.8 |
| 20MC-59A | Fish Oil | Intact for 60 mins | Ruptured in 5 |
| | | (No premature | minutes |
| | | releases) | |

In summary, pH dependent shell compositions containing methacrylic acid-ethyl acrylate copolymer inhibited the premature releases of fill materials in acid stage dissolution, even without curing the softgel capsules.

### Example 2—Addition of Organic Acids into the pH Dependent Shell Composition

Organic acids were added to wet gel masses of pH dependent shell composition to facilitate interaction between pectin and gelatin. Exemplary organic acids that were tested were lactic acid and tannic acid. Table 5 shows a wet gel composition for a pH dependent shell composition containing lactic acid.

TABLE 5
Dry Shell Composition of a pH Dependent Shell Composition Including
Lactic Acid (Compositions of Lots 20MC-58A and 20MC-58B)

| | wt % (based on total weight of dry shell |
|---|---|
| INGREDIENT | composition) |
| Gelatin | 40-70 |
| Glycerin | 20-55 |
| Pectin | 6-16 |
| Lactic Acid | 0.2-2.0 |
| Gellan gum | 0.1-2 |
| Dextrose | 0.02-2.0 |
| Water | 6-15 |
| Total | 100 |

Fish oil and polyethylene glycol 400 were encapsulated into pH dependent shell compositions having the dry shell composition of Table 5 and dried. After drying, the softgel capsules were subjected to two-stage dissolution test conducted on a USP Apparatus II with a paddle at 50 RPM, where in the first stage, the softgel capsules were in acid stage (0.1N HCl) for two hours (120 minutes), and in the second stage, the softgel capsules were in buffer stage (Buffer pH 6.8). The results are summarized in Table 6.

TABLE 6
Two Stage Dissolution Test Result on Softgel Capsules Having pH
Dependent Shell Composition of Table 5

| | | Dissolution T0 @50 RPM | |
|---|---|---|---|
| Lot No | Fill | 0.1N HCl | Buffer pH 6.8 |
| 20MC-58A | Fish Oil | Premature Release | Ruptured in 5 |
| | | Observed-Capsules | minutes |
| | | Survived for 120 | |
| | | mins | |
| 20MC-58B | Polyethylene glycol | Premature Release | Ruptured in 6 |
| | 400 | Observed-Capsules | minutes |
| | | Survived for 120 | |
| | | mins | |

Table 7 shows a dry shell composition for a pH dependent shell composition containing tannic acid.

TABLE 7
Dry Shell Composition of a pH Dependent Shell Composition Including
Tannic Acid (Compositions of Lots 20MC-65A and 20MC-65B)

| | wt % (based on total weight of dry shell |
|---|---|
| INGREDIENT | composition) |
| Gelatin | 40-75 |
| Glycerin | 20-45 |
| Pectin | 6-16 |
| Lactic Acid | 0.2-5 |
| Gellan gum | 0.3-2.5 |
| Dextrose | 0.01-2.0 |
| Water | 6-15 |
| Total | 100 |

Fish oil and polyethylene glycol 400 were encapsulated into pH dependent shell compositions having the dry shell mass composition of Table 7 and dried. After drying, the softgel capsules were subjected to two-stage dissolution test conducted on a USP Apparatus II with a paddle at 50 RPM, where in the first stage, the softgel capsules were in acid stage (0.1N HCl) for two hours (120 minutes), and in the second stage, the softgel capsules were in buffer stage (Buffer pH 6.8). The results are summarized in Table 8.

TABLE 8
Two Stage Dissolution Test Result on Softgel Capsules Having pH
Dependent Shell Composition of Table 7

| | | Dissolution T0 @50 RPM | |
|---|---|---|---|
| Lot No | Fill | 0.1N HCl | Buffer pH 6.8 |
| 20MC-65A | Fish Oil | Premature Release | Ruptured in 3 |
| | | Observed-Capsules | minutes |
| | | Survived for 120 | |
| | | mins | |
| 20MC-65B | Polyethylene glycol | Premature Release | Ruptured in 4 |
| | 400 | Observed-Capsules | minutes |
| | | Survived for 120 | |
| | | mins | |

Addition of organic acids to the pH dependent shell compositions improved the robustness of the shell compositions and correspondingly of the softgel capsules.

### Example 3 Washing pH Dependent Shell Composition with Organic Acids

Freshly manufactured wet fish oil capsules encapsulated using gel mass shown in Table 5 were washed with lactic acid and allowed to dry.

After drying the capsules were subjected to two stage dissolution tests conducted on a USP Apparatus II with a paddle at 50 RPM, where in the first stage, the softgel capsules were in acid stage (0.1N HCl) for two hours (120 minutes), and in the second stage, the softgel capsules were in buffer stage (Buffer pH 6.8). The results are summarized in Table 9.

TABLE 9
Two Stage Dissolution Test Result on Softgel Capsules
Having pH Dependent Shell Composition of Table 5
and Further Washed with Lactic Acid

| | | Dissolution T0 @50 RPM | |
|---|---|---|---|
| Lot No | Fill | 0.1N HCl | Buffer pH 6.8 |
| 20MC-58A* | Fish Oil | Intact for 120 | Ruptured in 8 |
| | | minutes (No | minutes |
| | | premature Release) | |

Treating pH dependent shell compositions with organic acids facilitated the interaction between pectin and gelatin and inhibited the premature release thereof. For simplicity of explanation, the embodiments of the methods of this disclosure are depicted and described as a series of acts. However, acts in accordance with this disclosure can occur in various orders and/or concurrently, and with other acts not presented and described herein. Furthermore, not all illustrated acts may be required to implement the methods in accordance with the disclosed subject matter. In addition, those skilled in the art will understand and appreciate that the methods could alternatively be represented as a series of interrelated states via a state diagram or events.

In the foregoing description, numerous specific details are set forth, such as specific materials, dimensions, processes parameters, etc., to provide a thorough understanding of the present invention. The particular features, structures, materials, or characteristics may be combined in any suitable manner in one or more embodiments. The words “example” or “exemplary” are used herein to mean serving as an example, instance, or illustration. Any aspect or design described herein as “example” or “exemplary” is not necessarily to be construed as preferred or advantageous over other aspects or designs. Rather, use of the words “example” or “exemplary” is intended to present concepts in a concrete fashion. As used in this application, the term “or” is intended to mean an inclusive “or” rather than an exclusive “or”. That is, unless specified otherwise, or clear from context, “X includes A or B” is intended to mean any of the natural inclusive permutations. That is, if X includes A; X includes B; or X includes both A and B, then “X includes A or B” is satisfied under any of the foregoing instances. Reference throughout this specification to “an embodiment”, “certain embodiments”, or “one embodiment” means that a particular feature, structure, or characteristic described in connection with the embodiment is included in at least one embodiment. Thus, the appearances of the phrase “an embodiment”, “certain embodiments”, or “one embodiment” in various places throughout this specification are not necessarily all referring to the same embodiment.

The present invention has been described with reference to specific exemplary embodiments thereof. The specification and drawings are, accordingly, to be regarded in an illustrative rather than a restrictive sense. Various modifications of the invention in addition to those shown and described herein will become apparent to those skilled in the art and are intended to fall within the scope of the appended claims.

1. A delayed release softgel capsule comprising:
(a) a fill material; and
(b) a pH dependent shell composition,
wherein the fill material comprises at least one active agent, and
wherein the pH dependent shell composition comprises gelatin, pectin, dextrose, and from about 0.5 wt. % to about 10 wt. % of a synthetic polymer, based on total weight of the dry pH dependent shell composition.

2. The delayed release softgel capsule of claim 1, wherein the pH dependent shell composition further comprises a plasticizer.

3. (canceled)

4. The delayed release softgel capsule of claim 1, wherein the pectin is selected from the group consisting of amidated pectin, non-amidated pectin and combinations thereof.

5. The delayed release softgel capsule of claim 1, wherein the pH dependent shell composition comprises about 40 wt % to about 80 wt % of a gelatin, based on the dry pH dependent shell composition weight.

6. The delayed release softgel capsule of claim 1, wherein the pH dependent shell composition comprises about 2 wt % to about 20 wt % of pectin, based on the dry pH dependent shell composition weight.

7. The delayed release softgel capsule of claim 1, wherein the pH dependent shell composition comprises about 0.01 wt % to about 4 wt % of dextrose, based on the dry pH dependent shell composition weight.

8. The delayed release softgel capsule of claim 2, wherein the pH dependent shell composition comprises about 15 wt % to about 40 wt % of a plasticizer, based on the dry pH dependent shell composition weight.

9.-11. (canceled)

12. The delayed release softgel capsule of claim 2, wherein the plasticizer is selected from the group consisting of glycerin, sorbitol and combinations thereof.

13. (canceled)

14. The delayed release softgel capsule of claim 1, wherein the synthetic polymer comprises a methacrylic acid-ethyl acrylate copolymer.

15. The delayed release softgel capsule of claim 1,
wherein the pH dependent shell composition does not dissolve at an acid stage pH of 1.2, 2.0, 3.0, 4.0, 5.0, 6.0, or a sub-range therein, for at least about 15 minutes, at least about 30 minutes, at least about 45 minutes, at least about 60 minutes, at least about 90 minutes, or at least about 120 minutes when measured with a USP Apparatus II with paddles at from about 50 RPM to about 250 RPM, from about 500 ml to about 900 ml 0.1N HCL adjusted to the acid stage pH with phosphate buffer solution, sodium hydroxide solution, or potassium hydroxide solution; and
wherein the pH dependent shell composition dissolves at a buffer pH of above about 6.5, above about 6.8, above about 7.0, above about 7.5, above about 8.0, or above about 8.5 in up to about 60 minutes, up to about 45 minutes, up to about 30 minutes, up to about 15 minutes, or up to about 10 minutes, when measured with a USP Apparatus II with paddles at from about 50 RPM to about 250 RPM, from about 500 ml to about 900 ml phosphate buffer solution, sodium hydroxide solution, or potassium hydroxide solution, adjusted to the buffer pH.

16. The delayed release softgel capsule of claim 1,
wherein the pH dependent shell composition does not disintegrate at an acid stage pH of 1.2, 2.0, 3.0, 4.0, 5.0, 6.0, or a sub-range therein, for at least about 15 minutes, at least about 30 minutes, at least about 45 minutes, at least about 60 minutes, at least about 90 minutes, or at least about 120 minutes when measured with a USP disintegration apparatus from about 500 ml to about 900 ml 0.1N HCL adjusted to the acid stage pH with phosphate buffer solution, sodium hydroxide solution, or potassium hydroxide solution; and
wherein the pH dependent shell composition disintegrated at a buffer pH of above about 6.5, above about 6.8, above about 7.0, above about 7.5, above about 8.0, or above about 8.5 in up to about 60 minutes, up to about 45 minutes, up to about 30 minutes, up to about 15 minutes, or up to about 10 minutes, when measured with a USP disintegration apparatus, from about 500 ml to about 900 ml phosphate buffer solution, sodium hydroxide solution, or potassium hydroxide solution, adjusted to the buffer pH.

17. (canceled)

18. (canceled)

19. A process of preparing a delayed release softgel capsule according to claim 1 comprising:
(a) preparing a fill material comprising an active agent; and
(b) encapsulating the fill material with a pH dependent shell composition.

20.-24. (canceled)

25. A method for tuning the pH dependent dissolution profile of a delayed release softgel capsule comprised of a fill material encapsulated in a pH dependent shell composition, the method comprising adjusting an amount of pectin and an amount of synthetic polymer in the pH dependent shell composition to attain a target pH dependent dissolution profile in acidic medium and/or in buffer medium.

26.-29. (canceled)

30. A method of reducing incidence of belching comprising, administering to a subject in need thereof a delayed release softgel capsule according to claim 1.

31. (canceled)

32. (canceled)

33. A delayed release softgel capsule comprising:
(a) a fill material; and
(b) a pH dependent shell composition,
wherein the fill material comprises at least one active agent, and
wherein the pH dependent shell composition comprises gelatin, pectin, dextrose, and an organic acid.

34.-52. (canceled)

53. A process of preparing a delayed release softgel capsule according to claim 33 comprising:
(a) preparing a fill material comprising an active agent;
(b) encapsulating the fill material with a pH dependent shell composition.

54.-60. (canceled)

61. A method for tuning the pH dependent dissolution profile of a delayed release softgel capsule comprised of a fill material encapsulated in a pH dependent shell composition, the method comprising adjusting an amount of pectin and an amount of organic acid in the pH dependent shell composition to attain a target pH dependent dissolution profile in acidic medium and/or in buffer medium.

62. (canceled)

63. (canceled)

64. (canceled)

65. A method of reducing incidence of belching comprising, administering to a subject in need thereof a delayed release softgel capsule according to claim 33.

66. (canceled)

67. (canceled)

68. A delayed release softgel capsule comprising:
(a) a fill material; and
(b) a pH dependent shell composition,
wherein the fill material comprises at least one active agent, and
wherein the pH dependent shell composition comprises a film former and from about 0.5 wt. % to about 10 wt. % of a synthetic polymer, based on total weight of the dry pH dependent shell composition.

69. (canceled)

70. The delayed release softgel capsules of claim 68, wherein the film former comprises a non-animal derived gelling agent comprising carrageenan, starch, pregelatinized starch, xanthan gum, agar, pectin, alginate, sugar, high molecular weight polyethylene glycol, sugar derived alcohol, a cellulose derivative, a cellulosic polymer, hydroxyethylcellulose, hydroxypropylcellulose, hydroxypropylmethylcellulose, carboxymethylcellulose, microcrystalline cellulose, attapulgite, bentonite, dextrin, alginate, kaolin, lecithin, magnesium aluminum silicate, carbomer, carbopol, silicon dioxide, curdlan, furcelleran, albumin, soy protein, chitosan, or a combination thereof.

71.-74. (canceled)

75. A delayed release softgel capsule comprising:
(a) a fill material; and
(b) a pH dependent shell composition,
wherein the fill material comprises at least one active agent, and
wherein the pH dependent shell composition comprises a film former and an organic acid.

76.-82. (canceled)
